SEQUENCE LISTING (139 sequences)

SEQ ID NO: 1 - protein, 1548 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLIDENNVLDATKTFVSLSLFNILRFPLTMLPMLITNLVQTQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQVLSKYLSGLALAIGGLHCSMNVFNKLLNTGLKWPMELFDTTPLGRILSRYSKDVDTVDSVLPAITVQLLNTCFGVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAFYSMAKDANLV

SEQ ID NO: 2 - protein, 1414 aa, Anopheles gambiae
MVEPVSLPLVDHLVVYGFQYLLVCLFFLSELSYVKQDVPVQTSRRTVHSIFSAFLVLVTLAGVVVAAFRLVDDSVAVWRDGIEAISLVGILFLQIYSIRRNVDHIYLFTFWTLRTLALSMDVAFDRTEWYDFMHLMLAFAWLCACGIRSYSGGGHDSSTGSNTPRKPNFIRGLFFSWMDSTYREAHRGSVAFYQGTLFQGTLPEDRRCEQLLELYEKANARRGYTAVDDGSGRMESELCRFTIGKLLSPFRGEIILAGLNRFVLISLFFLCPYLLRLLLEENQPRMYQKWIVTALFDASTIIAILNTHYQHTTQDIGLRIRSILMGAIYRSILHDGITSNASSDTLTSDTALFVPFIQNLHMMWSAPLIILITFVALWVGVLGPIGTVGLAIIVAVIAITRKLAKKIAAQEKHITAHSNDRVRLTTASIEQMQQIKSDLMEPFFEQRIGEHRRAELSHMCTYILYDALKYLLSIATPMIVACGTFLFMYVVGSGALLTVQSMFVAIALFGLTRYPLSELPNLMANWGTINVKLQVINEVVCSGKQRKSSGKMPQNGSSTGGAGRGSFEKMQEVVHTFVDQLEDSIADTSRAEVLRIERAKFSTEKNTILRGINLTLREGTFIGVSGTHGSGKTSLLRAMIGRLQRTGGTSTIAWNRVAYCPQTPWIHSGTIRSNILFGQEYEKSRYEEVLRACCLEEDLKTFPDYDERVVSEGGHSLSGGQARRVSLARAVYRHADVYLLDDPLRSLDPNVARKVFEGVFHRQHGLLAGCTCVFISHDPEHLSIADKVLVMAGGTIEKVLKPAEVSVELLGQLNGAEDEPEQEEKAPKADQQNKKRARKAQPKGTDHGEGNVSLGLYVTFARMLKRRYCVGALCFESTVTALDIVIITLLAQWAASDKQTSGALLHTTWILCVWVLLIFLKTAIIHWAGLSLSKRVHSQMLATILRQPMEFFDLNDSGVIVNRFSNDLKVVDKTIITSVRSVLSASFSVLGTLMLFVYKLHSKLLLFVLAFTAALMLVCGLKRLLSYHLQVARTLKRFEASSRSPIILQYNETIQGIDTIKAYEAEDRLLRQFFERVDTHQNYIYHNRFANRWIGIRLEFIGAIVIYYVALLTVSNQSMVGFAFVGIIVSYVLRLIPSLNSLLLALGALEENIISFERVAQYLDLQRETNDDTGVDYPTSGMDKHPVLGPIIYRDFSLTHADGSTVLHNVTLTIAAGEKLGIVGRTGSGKSSFIGTLFRFYPKHTTGYISIAHVELGRISLQKLRGELTLVPQSTSLFSGVVQNFIDPRNGHTDEELIRCLRECGLGNVHLATPLENLSVGQCQLLCLVRGFLRKKPIIILDEATSALDEATEDLILKVLDKQFHGRTVLMIAHHLNTLRNCHRVLWLQEGRVRKIAPLQDYTVEERAELGFRD

SEQ ID NO: 3 - protein, 1499 aa, Anopheles gambiae
MTFEEFCGGPFWDDDLTWREEDPDLTFCFQRVILQWTPCFFLFVFSMYEVLRIVTSRYRDIPWNWFNITKMIFTFALMVMSWVDLGVGLANDWIESLLSMLPSLPFQIMAMALYFFYRKYGIRSTGTMFIFWFLKAFFGIIQMRTEAMLHDVRGSGTGDFAEFQFVSYTIQYTFVCCVLLLELFPDKEPRYSEWAKLKNPNPELRSSFFSRLFYLYFDSYAWRGFRKPLTDDDMYDLNPEDTSRALVPPFDKYWYESVEKGRRKQIAADKKAGKTNLVYKPNAATNGSVLPAMVKAYGGPFWFAGMLQFAISGLQFASPYLMQEIMAVIALDGPFWKGMIITLGLFLTSLLIALFNGQYFHRTFLVGFRIRTGLISAIYRKALRISSFAKKDTTVGEIVNLMAVDAQRFFELTSYLHVLWSAPLIIALCIYLLYELLGPAVFAGLGVMVIMIPITGFIATRMRDLQVEQMKIKDERVKKMNEILGGIKVLKLYAWEPSFQDTVVTVRNEELDVLKSAAYYGAGTYFVWTMAPFLVTLASFAVYVMIDEENVLDPQTAFVALALFNILRFPLAMFPMMITFAMQAWVSIKRIDKFMNSEELDPNNVTHNKSENALEVKDGTFSWGDDAPTLKNINLALRRGKLSAVVGGVGTGKSSLISALLGEMEKMKGSVNTDGSIAYVPQQAWIQNATLRDNILFGRPFDQAKYDKVIECCALRPDLEMLPGGDTTEIGEKGINLSGGQKQRVALARAVYADSEVYLFDDPLSAVDAHVGKHIFEKVIGPSGMLVGRSRLLVTHGISFLPFVEEIFVMKDGEVSESGSYQELLDQKGAFAEFLTQHIQEMDDEDEDELKLIQEALKDGEAKKIVQRAMSTRSQRSGSSNGSVRKKRVSRAESRNSNKPRAVEQTVAQQSSATLIEKEESATGAVGYVVYIKYFKGIGLWLGFWSIFFSVINQGASIYANIWLTDWSEDPEAATDPSVRDMYLGVYGGLGGAQSIALLIASVTLALGCIKAARELHNNLLESSMRMPMSFFDTTPLGRIMNRFSKDVDVVDNILPQSIRAWLLMFFNVIGVFVVIGISTPIFLAVVPAFLVIYYLIQKFYIATSRQLKRLESVTRSPIYSHFGESITGQSTIRAYGQQDRFMNESEQRVDYNQLTSYPSIIANRWLAVRLELVGALVVFFAALFAMVARDTIGQATVGLSISYALQISATLSFLVRMTAEVETNIVAIERLEEYTVLPREAEWQLGHVDKAWPVEGKVEFKDYQIRYREGLDLVIRGISLNVRGGEKIGIVGRTGAGKSSLTLGLFRIVEAAGGQIIIDGLDISKMGLHQLRGRLTIIPQDPVLFSGTLRANVDPFKSYSDDLVWKALELSHLKTFVKGLAAGLDHEIAENGENLSVGQRQLICLARAVLRKTKVLILDEATAAVDLETDDLIQKTIRTEFADCTILTIAHRLNTILDSDRVLVLDKGLVAECDSPQNLLANRESIFFGMAKNAGIVS

SEQ ID NO: 4 - protein, 1505 aa, Anopheles gambiae
MTFEDFCGGPFWDGEFVWDVDNPNLTFCFQRVILQWVPCLFLFVFSIYDIFKITESKYRDIPWNWYNLSKMLVIFLLMCMCWIDLGMVVGYQDEQGLYDVQILTAVFNALAYIDLLVLLFFMRKYGVRTSGTMFMFWFLRMFFGIIQLRTEVMENDKRPNAIGSGDTVDFWEYQYVSYILQYSLICLMLVLELFPDKEPSFSYYPKAAKPNPELRSSFFSKLLFLHFDAFAWKGFRNPLTMNDMYDINPQDSARELVPPFDKYWKISVEKGRKQQMASDRKAGKPDIDYKPHSPSNGSVLYTMIRAYGGPFWFAGMLQLAISGLQFASPYLMQELMAVIAFDGPLWKGFLLTFGLFGASLLLGLFNGQYLFYTFLSGFRIRTGLISAIYRKALRISSAAKKDTTVGEIVNLMAVDAQKFFELTSYLHILWSALLIIGLCVFLLYDILGPAVFAGLGVMILMTPVSGVVAAKLKTHQVAQMKLKDERVKKMNEILGGIKVLKLYAWEPSFQDSILNVRDEEVGILKKMAYYGAGIFFTFTIAPFLVTLVSFAVYVLIDENNVLDPQTAFVSLALFNIMRFPLGMFPMVVTFSMQAWVSIKRIDKFLNSAELDPNNVTHNKSDEALTIKDGTFSWGDETPTLKNINLSLRKGQLSAIVGTVGTGKSSLISALLGEMEKISGHVNTDGSIAYVPQQAWIQNATLRDNILFGKAFDQRKYDNVIECCALRPDLEMLPGGDSTEIGEKGINLSGGQKQRVALARAVYADAEVYLFDDPLSAVDAHVGKHIFEKVIGPSGMLVGKSRLLVTHGISFLPFVENILVLKDGEISESGTYQELIDQKGAFAEFLSQHIQELDDEDEEISLIQETLNDGVVNNVIQRALSVRSNRSNGSDGSTRKKPISRQVSKQSVHSKTVTTVPGRANLIGVEESATGAVTWLVYKKYIQSIGFKFGFGSVLFTAINQGSGIFSNLWLTDWSEDPDAATDPSVRDKYLGVYGALGGAQSIALFVAALLISLGCLKAAKESHNKLLESCLRMPMSFFDTTPLGRIINRFSKDVDVVDNVLPVTIRAWLLFLFNVFGVFIVIGTSTPIFLAVVPPLMVIYYFVQRFYIDTSRQLKRLESVTRSPIYSHFGESIGGQSTIRAYGQQDRFTQESERRVDYNQLVSYPTIVANRWLAVRLELIGSCVILFAALFAILARDTIGQATVGVSISYALQISHYLSFLVRMTSEVETNIVAVERLEEYTVLPREAEWQKGTVDKAWPVEGKVEFKDYQIRYREGLDLVIRGISLNVRGGEKIGIVGRTGAGKSSLTLGLFRIVEAAGGQIIIDGLDISKMGLHQLRGRLTIIPQDPVLFSGTLRANVDPFKSYSDDQVWKALELSHLKTFVKGLSAGLDHEIAENGENLSVGQRQLICLARAVLRKTKVLILDEATAAVDLETDDLIQKTIRTEFADCTILTIAHRLNTILDSDRVLVLDKGLVAECDSPQNLLANRESIFYGMAKNAGIVS

SEQ ID NO: 5 - protein, 1507 aa, Anopheles gambiae
MTFEDFCGGPFWDDEFVWDVDNPNLTFCFQRVILQWVPCLFLFVFSIYDIFKITESKYRDIPWNWYNLSKMLVIFLLMCMCWIDLGMVVGYQDEQGLYDVQILTAVFNALAYIDLLVLLFFMRKYGVRTSGTMFMFWFLRMFFGIIQLRTEVMENDKRPNAIGSGDTVDFWEYQYVSYILQYSLICLMLVLELFPDKEPTFSYYPKSKNPNPELKSSFFAKLLFLYFDTFAWKGFRKPLTMEEMYDINPQDTSRELVPPFDKYWDMSVANGRKKQIAADKKAGKTNIEYKPHSETNGSSLYAMVRAYGAPFWFAGMLQLAISGLQFASPYLMQEMMAVIALDGPVWKGLLLTFALFAASLLLALLNGQYYYNTFLSGFRIRTGLVSAIYRKALRISSAAKKDTTVGEIVNLMAVDAQRFFELTSYMHILWSGVLIIALCVYLLYDILGAAVFAGLGVMILITPVSGVIATKMRDAQVAQMKIKDDRVKKMNEILGGIKVLKLYAWEPSFQDNILTVRKEEIGILKRMAYYGAGIYFTFTIAPFLVTLVSFAVYVLMDEENILDPQTAFVSLALFNILRFPLGMLPMMVTFSMQAWVSVKRIDKFLNSAELDPSNVSNNKSDEALTIKDGTFSWGDETPTLKNINLSLRKGQLSAIVGTVGTGKSSLISALLGEMEKISGHVNTDGSIAYVPQQAWIQNATLRDNILFGKAFDQRKYDNVIECCALRPDLEMLPGGDSTEIGEKGINLSGGQKQRVALARAVYADAEVYLFDDPLSAVDAHVGKHIFEKVIGPSGMLVGKSRLLVTHGISYLPFVENIFVVKDGEISESGSYQQLLDQKGAFAEFLTQHIQELDEADEDEIKLIQETLKDETAQRIVERSLSVRSGRSGGSNSSIRKKRISRQESKASAKKEDPIIQNLDKATLIEKEESATGAVTWAVYKKYVTAIGFQFGFWSVVFSAINQGSGIYSSMWLTDWSEDPEAITDTSVRDMYLGVYGALGGVQSIALFIGSVLLALGCLKAAEESHNKLLESSMHMPMSFFDTTPLGRIINRFSKDVDVVDNILPATIRAWLLMLFSVIGVFVVIGISTPIFLAIVPPLMIIYYFVQRFYIETSRQLKRLESVTRSPIYSHFGESIGGQSTIRAYAQQERFIRESEHRVDYNQLVTYPTILANRWLGVRLEIIGSLVILFAALFAILARDTIGQATVGLSISYALQISNVLSFLVRMTAEVETNIVAIERLEEYTVLPREAEWQKGTVDKAWPVEGKVEFKDYQIRYREGLDLVIRGISLNVRGGEKIGIVGRTGAGKSSLTLGLFRIVEAAGGQIIIDGLDISKMGLHQLRSRLTIIPQDPVLFSGTLRANVDPFKSYSDDQVWKALELSHLKTFVKGLTAGLDHEIAENGENLSVGQRQLVCLARAILRKTKVLILDEATAAVDLETDDLIQKTIRTEFADCTILTIAHRLNTILDSDRVLVLDKGLVAECDSPQNLLANRDSIFHSMAKNAGIVS

SEQ ID NO: 6 - protein, 1531 aa, Homo sapiens
MALRGFCSADGSDPLWDWNVTWNTSNPDFTKCFQNTVLVWVPCFYLWACFPFYFLYLSRHDRGYIQMTPLNKTKTALGFLLWIVCWADLFYSFWERSRGIFLAPVFLVSPTLLGITTLLATFLIQLERRKGVQSSGIMLTFWLVALVCALAILRSKIMTALKEDAQVDLFRDITFYVYFSLLLIQLVLSCFSDRSPLFSETIHDPNPCPESSASFLSRITFWWITGLIVRGYRQPLEGSDLWSLNKEDTSEQVVPVLVKNWKKECAKTRKQPVKVVYSSKDPAQPKESSKVDANEEVEALIVKSPQKEWNPSLFKVLYKTFGPYFLMSFFFKAIHDLMMFSGPQILKLLIKFVNDTKAPDWQGYFYTVLLFVTACLQTLVLHQYFHICFVSGMRIKTAVIGAVYRKALVITNSARKSSTVGEIVNLMSVDAQRFMDLATYINMIWSAPLQVILALYLLWLNLGPSVLAGVAVMVLMVPVNAVMAMKTKTYQVAHMKSKDNRIKLMNEILNGIKVLKLYAWELAFKDKVLAIRQEELKVLKKSAYLSAVGTFTWVCTPFLVALCTFAVYVTIDENNILDAQTAFVSLALFNILRFPLNILPMVISSIVQASVSLKRLRIFLSHEELEPDSIERRPVKDGGGTNSITVRNATFTWARSDPPTLNGITFSIPEGALVAVVGQVGCGKSSLLSALLAEMDKVEGHVAIKGSVAYVPQQAWIQNDSLRENILFGCQLEEPYYRSVIQACALLPDLEILPSGDRTEIGEKGVNLSGGQKQRVSLARAVYSNADIYLFDDPLSAVDAHVGKHIFENVIGPKGMLKNKTRILVTHSMSYLPQVDVIIVMSGGKISEMGSYQELLARDGAFAEFLRTYASTEQEQDAEENGVTGVSGPGKEAKQMENGMLVTDSAGKQLQRQLSSSSSYSGDISRHHNSTAELQKAEAKKEETWKLMEADKAQTGQVKLSVYWDYMKAIGLFISFLSIFLFMCNHVSALASNYWLSLWTDDPIVNGTQEHTKVRLSVYGALGISQGIAVFGYSMAVSIGGILASRCLHVDLLHSILRSPMSFFERTPSGNLVNRFSKELDTVDSMIPEVIKMFMGSLFNVIGACIVILLATPIAAIIIPPLGLIYFFVQRFYVASSRQLKRLESVSRSPVYSHFNETLLGVSVIRAFEEQERFIHQSDLKVDENQKAYYPSIVANRWLAVRLECVGNCIVLFAALFAVISRHSLSAGLVGLSVSYSLQVTTYLNWLVRMSSEMETNIVAVERLKEYSETEKEAPWQIQETAPPSSWPQVGRVEFRNYCLRYREDLDFVLRHINVTINGGEKVGIVGRTGAGKSSLTLGLFRINESAEGEIIIDGINIAKIGLHDLRFKITIIPQDPVLFSGSLRMNLDPFSQYSDEEVWTSLELAHLKDFVSALPDKLDHECAEGGENLSVGQRQLVCLARALLRKTKILVLDEATAAVDLETDDLIQSTIRTQFEDCTVLTIAHRLNTIMDYTRVIVLDKGEIQEYGAPSDLLQQRGLFYSMAKDAGLV

SEQ ID NO: 7 - protein, 49 aa, Drosophila melanogaster
VSLVTFATYVLTSEANQLSVEKVLVSIALFDLMKLPLTILPMLSVDIAE

SEQ ID NO: 8 - protein, 49 aa, Drosophila melanogaster
VSLVTFATYVLIDENNVLDATKTFVSLSLFNILRFPLTMLPMLITNLVQ

SEQ ID NO: 9 - protein, 73 aa, Drosophila melanogaster
ATNFFSSLAISLGCLKCSQLLHQTLLYYNLRWPMELFDTTPLGRIVNRFSKDIDTIDNVLPFNIRVVIGQAYM

SEQ ID NO: 10 - protein, 73 aa, Drosophila melanogaster
VTGYLSTLILSLGCVYSARYMHNVLLHGTLRWPMEMFDITPLGRIVNRFSKDVDTIDNTLPLNLRVVILQLFA

SEQ ID NO: 11 - protein, 73 aa, Drosophila melanogaster
TSYFFCSLTLALGCIPCSKVLHETLLSYVFRWPMELFDTTPLGRVVNRFSKDVDTIDNVLPMLWRMVISQAFA

SEQ ID NO: 12 - protein, 73 aa, Drosophila melanogaster
LSKYLSGLALAIGGLHCSMNVFNKLLNTGLKWPMELFDTTPLGRILSRYSKDVDTVDSVLPAITVQLLNTCFG

SEQ ID NO: 13 - protein, 74 aa, Drosophila melanogaster
VLAYFAVVIVYLGGFQAAKTIHNELLAVIIRGSVCRFFDITPIGRLLNSFSGDMDVVDEELPATMDSFMTFIFM

SEQ ID NO: 14 - protein, 73 aa, Drosophila melanogaster
LCNYGAAISLFTATLHASSRVFHRLFNNIMHCPSEFFDTTPKGRILDRCSSDVNCLDLVMPLNIRMVMSTAFQ

SEQ ID NO: 15 - protein, 73 aa, Drosophila melanogaster
FTSFFSDLAPALGSLHAAKVLHSMLLENVLRAPMTMFDTTPVGRILSRFSKDVESVDQKMPQVINDCIWCAFE

SEQ ID NO: 16 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLTSEANQLSVEKVLVSIALFDLMKLPLTILPMLSVDIAETQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQVATNFFSSLAISLGCLKCSQLLHQTLLYYNLRWPMELFDTTPLGRIVNRFSKDIDTIDNVLPFNIRVVIGQAYMVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 17 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLTSEANQLSVEKVLVSIALFDLMKLPLTILPMLSVDIAETQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQVVTGYLSTLILSLGCVYSARYMHNVLLHGTLRWPMEMFDITPLGRIVNRFSKDVDTIDNTLPLNLRVVILQLFAVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 18 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLTSEANQLSVEKVLVSIALFDLMKLPLTILPMLSVDIAETQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQVTSYFFCSLTLALGCIFCSKVLHETLLSYVFRWPMELFDTTPLGRVVNRFSKDVDTIDNVLPMLWRMVISQAFAVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 19 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLTSEANQLSVEKVLVSIALFDLMKLPLTILPMLSVDIAETQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQVLSKYLSGLALAIGGLHCSMNVFNKLLNTGLKWPMELFDTTPLGRILSRYSKDVDTVDSVLPAITVQLLNTCFGVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 20 - protein, 1539 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLTSEANQLSVEKVLVSIALFDLMKLPLTILPMLSVDIAETQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQGVLAYFAVVIVYLGGFQAAKTIHNELLAVIIRGSVCRFFDITPIGRLLNSFSGDMDVVDEELPATMDSFMTFIFMVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 21 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLTSEANQLSVEKVLVSIALFDLMKLPLTILPMLSVDIAETQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQGLCNYGAAISLFTATLHASSRVFHRLFNNIMHCPSEFFDTTPKGRILDRCSSDVNCLDLVMPLNIRMVMSTAFQVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 22 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLTSEANQLSVEKVLVSIALFDLMKLPLTILPMLSVDIAETQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQGFTSFFSDLAPALGSLHAAKVLHSMLLENVLRAPMTMFDTTPVGRILSRFSKDVESVDQKMPQVINDCIWCAFEVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 23 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLIDENNVLDATKTFVSLSLFNILRFPLTMLPMLITNLVQTQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQVATNFFSSLAISLGCLKCSQLLHQTLLYYNLRWPMELFDTTPLGRIVNRFSKDIDTIDNVLPFNIRVVIGQAYMVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 24 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLIDENNVLDATKTFVSLSLFNILRFPLTMLPMLITNLVQTQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQVVTGYLSTLILSLGCVYSARYMHNVLLHGTLRWPMEMFDITPLGRIVNRFSKDVDTIDNTLPLNLRVVILQLFAVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 25 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLIDENNVLDATKTFVSLSLFNILRFPLTMLPMLITNLVQTQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQVTSYFFCSLTLALGCIFCSKVLHETLLSYVFRWPMELFDTTPLGRVVNRFSKDVDTIDNVLPMLWRMVISQAFAVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 26 - protein, 1539 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLIDENNVLDATKTFVSLSLFNILRFPLTMLPMLITNLVQTQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQGVLAYFAVVIVYLGGFQAAKTIHNELLAVIIRGSVCRFFDITPIGRLLNSFSGDMDVVDEELPATMDSFMTFIFMVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 27 - protein, 1538 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLIDENNVLDATKTFVSLSLFNILRFPLTMLPMLITNLVQTQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQGLCNYGAAISLFTATLHASSRVFHRLFNNIMHCPSEFFDTTPKGRILDRCSSDVNCLDLVMPLNIRMVMSTAFQVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAF

SEQ ID NO: 28 - protein, 1548 aa, Drosophila melanogaster
MADDTSSPMDRFCGSTFWNATETWYTNDPDFTPCFEQTALVWTPCAFYWAFVIFDFYYLKASLDRNIPWNKLNVSKALVNLGLLVITALDLIMALVKKGGDSELPLYDLDVWGPIIKFATFLLLFIFIPLNRKYGVQTTGCQFIFWFLLTVLSIPRCRTEVRLDAERQKILNSQQPSEQDFSWEEYQFVSFFIFFTFTSIMLILNCFADGMPRQTKYQRGENEIPELSASFLSRITYQWFDKMALKGYRNPLEEKDLWDLRPQDSCSEVMPIFAHHWNQNVRKNYKNKARVEPKAQFSNGNVTFENPHGEKNGRKKGMASIMPPIYKSFGGVFLFGALMKLFTDTLTFAQPQVLSLIISFVEAQDAEPEWKGILYAVLLFVLAAAQTFILGQYFHRMFIVGLRIRTALINAIYRKALRISNSTKKESTVGEIVNLMAVDAQRFMELTTYLNMIWSAPLQIGLALYFLWQQLGPSVLAGLAVMIILIPVNGVIASRIKTYQIRQMKYKDERVKLMNEVLSGIKVLKLYAWEPSFEKQVLDIRDKEIATLRSTAYLNAGTSFLWSCAPFLVSLVTFATYVLIDENNVLDATKTFVSLSLFNILRFPLTMLPMLITNLVQTQVSVNRINKFLNSEELDPNSVLHDSSKPHPMSIENGEFSWGDEITLRNINIEVKKGSLVALVGTVGSGKSSVVQAFLGEMEKLAGVVNTVGKLAYVPQQAWIQNATVRDNILFGQTYDRKRYNKVIDACALRADIDILSAGDLTEIGEKGINLSGGQKQRISLARAVYSDADLYLLDDPLSAVDAHVGKHIFEEVIGPKGILARKSRVLVTHGVTFLPQVDSIYVIKMGEISESGTFDQLVKNKGAFADFIIQHLQEGNEEEEELNQIKRQISSTADVPELLGTVEKAIKLARTESLSDSISVTSADSLMGGGGSLRRRTKRQDSHDSVASAASLKKKQEVEGKLIETEKSQTGGVEFAVYKHYIKSVGIFLSVATLVLNFVFQAFQIGSNLWLTQWANDQNVANDTGLRDMYLGVYGAFGFGQGFTSFFSDLAPALGSLHAAKVLHSMLLENVLRAPMTMFDTTPVGRILSRFSKDVESVDQKMPQVINDCIWCAFEVLATIVVISLSTPIFLAVIVPIAFLYYFAQRFYVATSRQLMRLESVSRSPIYSHFSETVTGASTIRAYNVGDRFIEESDAKVDKNQVCKYPSVIANRWLAIRLEMVGNLIILFASLFAVLGGQTNPGLVGLSVSYALQVTQTLNWLVRMSSDIETNIVSVERIKEYGETKQEAPWELEQDKNKPKNWPQEGRVEFQNFQVRYREGLDLVLRGVSFNIQGGEKVGIVGRTGAGKSSLTLALFRIIEAAGGRISIDGVDIASMGLHMLRSRLTIIPQDPVLFSGSLRINLDPFEIKTDDEIWKALELSHLKSFVKSLAAGLNHEIAEGGENLSVGQRQLVCLARALLRKTKVLVLDEATAAVDLETDDLIQKTIRTEFKECTVLTIAHRLNTILDSDKVIVLDKGQIIEFASPTELLDNPKSAFYSMAKDANLV

SEQ ID NO: 29 - DNA, 20 bp, Artificial Sequence
  Description of Artificial Sequence Synthetic primer
agtgattgccagtcgcatca

SEQ ID NO: 30 - DNA, 20 bp, Artificial Sequence
  Description of Artificial Sequence Synthetic primer
gccgttctcaatgctcattg

SEQ ID NO: 31 - DNA, 20 bp, Artificial Sequence
  Description of Artificial Sequence Synthetic primer
ctcggctatgtcaacactca

SEQ ID NO: 32 - DNA, 20 bp, Artificial Sequence
  Description of Artificial Sequence Synthetic primer
ttgcaccaggttggtgatca

SEQ ID NO: 33 - DNA, 18 bp, Artificial Sequence
  Description of Artificial Sequence Synthetic primer
aacgatcaaaatgtcgcc

SEQ ID NO: 34 - DNA, 19 bp, Artificial Sequence
  Description of Artificial Sequence Synthetic primer
cacgaatagtcgatgctcc

SEQ ID NO: 35 - DNA, 24 bp, Artificial Sequence
  Description of Artificial Sequence Synthetic primer
gggaattcgcgtggacagactaat

SEQ ID NO: 36 - DNA, 25 bp, Artificial Sequence
  Description of Artificial Sequence Synthetic primer
gggaattcgcgacgaacttcttctc

SEQ ID NO: 37 - DNA, 27 bp, Artificial Sequence
  Description of Artificial Sequence Synthetic primer
gggaattcttacctcgtacttcttttg

SEQ ID NO: 38 - DNA, 27 bp, Artificial Sequence
  Description of Artificial Sequence Synthetic primer
gggaattcttgttacagggtatctatc

SEQ ID NO: 39 - DNA, 29 bp, Artificial Sequence
  Description of Artificial Sequence Synthetic primer
gggaattcctatccaaatatttatcgggg

SEQ ID NO: 40 - DNA, 27 bp, Artificial Sequence
  Description of Artificial Sequence Synthetic primer
gggaattcgtttcacgtcattcttttc

SEQ ID NO: 41 - DNA, 27 bp, Artificial Sequence
  Description of Artificial Sequence Synthetic primer
gggaattcgtctttgcaattacggcgc

SEQ ID NO: 42 - DNA, 27 bp, Artificial Sequence
  Description of Artificial Sequence Synthetic primer
gggaattcgtgtgctagcctactttgc

SEQ ID NO: 43 - DNA, 18 bp, Artificial Sequence
  Description of Artificial Sequence Synthetic primer
ggagcgattgtgatttac

SEQ ID NO: 44 - DNA, 18 bp, Artificial Sequence
  Description of Artificial Sequence Synthetic primer
ctggccaacggaaaggtt

SEQ ID NO: 45 - DNA, 20 bp, Artificial Sequence
  Description of Artificial Sequence Synthetic primer
ggtcgagttcaaggactatc

SEQ ID NO: 46 - DNA, 20 bp, Artificial Sequence
  Description of Artificial Sequence Synthetic primer
tgagtcacactccgctacca

SEQ ID NO: 47 - DNA, 18 bp, Artificial Sequence
  Description of Artificial Sequence Synthetic primer
ggagctctggttgtgttc

SEQ ID NO: 48 - DNA, 18 bp, Artificial Sequence
  Description of Artificial Sequence Synthetic primer
ggaagttgtgtcatcttg

SEQ ID NO: 49 - DNA, 18 bp, Artificial Sequence
  Description of Artificial Sequence Synthetic primer
ggcagcttggtgatcctg

SEQ ID NO: 50 - DNA, 17 bp, Artificial Sequence
  Description of Artificial Sequence Synthetic primer
tgaccgaccgagaggtt

SEQ ID NO: 51 - DNA, 20 bp, Artificial Sequence
  Description of Artificial Sequence Synthetic primer
cacacagcatttttacagct

SEQ ID NO: 52 - DNA, 20 bp, Artificial Sequence
  Description of Artificial Sequence Synthetic primer
tctttcattgccgccgtatt

SEQ ID NO: 53 - DNA, 22 bp, Artificial Sequence
  Description of Artificial Sequence Synthetic primer
gatccgtttatttccttgccgc

SEQ ID NO: 54 - DNA, 21 bp, Artificial Sequence
  Description of Artificial Sequence Synthetic primer
tccagggcagtgattaccagt

SEQ ID NO: 55 - DNA, 12 bp, Drosophila melanogaster
gaacagaacgca

SEQ ID NO: 56 - DNA, 12 bp, Drosophila melanogaster
acataggtgctc

SEQ ID NO: 57 - DNA, 12 bp, Drosophila melanogaster
acaaaggtttcc

SEQ ID NO: 58 - DNA, 12 bp, Drosophila melanogaster
ttttaggtttca

SEQ ID NO: 59 - DNA, 12 bp, Drosophila melanogaster
gaatagacgcaa

SEQ ID NO: 60 - DNA, 12 bp, Drosophila melanogaster
atacagcccatc

SEQ ID NO: 61 - DNA, 12 bp, Drosophila melanogaster
ttttagctccgt

SEQ ID NO: 62 - DNA, 12 bp, Drosophila melanogaster
ttctagtcgcga

SEQ ID NO: 63 - DNA, 12 bp, Drosophila melanogaster
tcgaagttgtta

SEQ ID NO: 64 - DNA, 12 bp, Drosophila melanogaster
ttccagttacct

SEQ ID NO: 65 - DNA, 12 bp, Drosophila melanogaster
atgcagtgctat

SEQ ID NO: 66 - DNA, 12 bp, Drosophila melanogaster
tcccaggtgtgc

SEQ ID NO: 67 - DNA, 12 bp, Drosophila melanogaster
agctaggtcttt

SEQ ID NO: 68 - DNA, 12 bp, Drosophila melanogaster
tcgcaggtttca

SEQ ID NO: 69 - DNA, 12 bp, Drosophila melanogaster
ggttaggttctg

SEQ ID NO: 70 - DNA, 12 bp, Drosophila melanogaster
cttcagctttat

SEQ ID NO: 71 - DNA, 12 bp, Drosophila melanogaster
atttagaataat

SEQ ID NO: 72 - DNA, 12 bp, Drosophila melanogaster
ctatagaaaacc

SEQ ID NO: 73 - DNA, 12 bp, Drosophila melanogaster
ttctgggtgagt

SEQ ID NO: 74 - DNA, 12 bp, Drosophila melanogaster
attaaggtgagt

SEQ ID NO: 75 - DNA, 12 bp, Drosophila melanogaster
ttcctggtaaga

SEQ ID NO: 76 - DNA, 12 bp, Drosophila melanogaster
gccgaggtacag

SEQ ID NO: 77 - DNA, 12 bp, Drosophila melanogaster
gtgcaagtaagt

SEQ ID NO: 78 - DNA, 12 bp, Drosophila melanogaster
ctaaacgtaaga

SEQ ID NO: 79 - DNA, 12 bp, Drosophila melanogaster
ttccatgtaagt

SEQ ID NO: 80 - DNA, 12 bp, Drosophila melanogaster
gccaaggtaagt

SEQ ID NO: 81 - DNA, 12 bp, Drosophila melanogaster
tatatggtaatt

SEQ ID NO: 82 - DNA, 12 bp, Drosophila melanogaster
tttgcggtaatt

SEQ ID NO: 83 - DNA, 12 bp, Drosophila melanogaster
tttgcggtaaat

SEQ ID NO: 84 - DNA, 12 bp, Drosophila melanogaster
ttcggggtaaag

SEQ ID NO: 85 - DNA, 12 bp, Drosophila melanogaster
tttatggtattt

SEQ ID NO: 86 - DNA, 12 bp, Drosophila melanogaster
tttcaggtaatc

SEQ ID NO: 87 - DNA, 12 bp, Drosophila melanogaster
ttcgaggtaatt

SEQ ID NO: 88 - DNA, 12 bp, Drosophila melanogaster
agatcggtatgt

SEQ ID NO: 89 - DNA, 12 bp, Drosophila melanogaster
gttcaggtaagc

SEQ ID NO: 90 - DNA, 12 bp, Drosophila melanogaster
attcaggtgggt

SEQ ID NO: 91 - DNA, 12 bp, Anopheles gambiae
gtacaggtggac

SEQ ID NO: 92 - DNA, 12 bp, Anopheles gambiae
cttcaggtgggc

SEQ ID NO: 93 - DNA, 12 bp, Anopheles gambiae
atatagattact

SEQ ID NO: 94 - DNA, 12 bp, Anopheles gambiae
ttacaggacgat

SEQ ID NO: 95 - DNA, 12 bp, Anopheles gambiae
tttcagattatg

SEQ ID NO: 96 - DNA, 12 bp, Anopheles gambiae
ctctagggaact

SEQ ID NO: 97 - DNA, 12 bp, Anopheles gambiae
ttccagaattgt

SEQ ID NO: 98 - DNA, 12 bp, Anopheles gambiae
acacagaaaaca

SEQ ID NO: 99 - DNA, 12 bp, Anopheles gambiae
atttagatcgac

SEQ ID NO: 100 - DNA, 12 bp, Anopheles gambiae
ttatagagaact

SEQ ID NO: 101 - DNA, 12 bp, Anopheles gambiae
ttttagggaact

SEQ ID NO: 102 - DNA, 12 bp, Anopheles gambiae
tttcagaaatat

SEQ ID NO: 103 - DNA, 12 bp, Anopheles gambiae
atctagaattgt

SEQ ID NO: 104 - DNA, 12 bp, Anopheles gambiae
ttacagaaaaca

SEQ ID NO: 105 - DNA, 12 bp, Anopheles gambiae
atttagatcgac

SEQ ID NO: 106 - DNA, 12 bp, Anopheles gambiae
tcgcagagaaat

SEQ ID NO: 107 - DNA, 12 bp, Anopheles gambiae
tttcagacaact

SEQ ID NO: 108 - DNA, 12 bp, Anopheles gambiae
caccagaaatta

SEQ ID NO: 109 - DNA, 12 bp, Anopheles gambiae
ttccagaattgt

SEQ ID NO: 110 - DNA, 12 bp, Anopheles gambiae
ccacagaaaaca

SEQ ID NO: 111 - DNA, 12 bp, Anopheles gambiae
cccttggtgaga

SEQ ID NO: 112 - DNA, 12 bp, Anopheles gambiae
tccttggtaagc

SEQ ID NO: 113 - DNA, 12 bp, Anopheles gambiae
gctgaggtaagt

SEQ ID NO: 114 - DNA, 12 bp, Anopheles gambiae
ttttgggtaagt

SEQ ID NO: 115 - DNA, 12 bp, Anopheles gambiae
gcttatgtaagt

SEQ ID NO: 116 - DNA, 12 bp, Anopheles gambiae
ataccagtaagt

SEQ ID NO: 117 - DNA, 12 bp, Anopheles gambiae
cttcaggtatgt

SEQ ID NO: 118 - DNA, 12 bp, Anopheles gambiae
attcaggtaaga

SEQ ID NO: 119 - DNA, 12 bp, Anopheles gambiae
gcttatgtgagt

SEQ ID NO: 120 - DNA, 12 bp, Anopheles gambiae
gatgcagtaagt

SEQ ID NO: 121 - DNA, 12 bp, Anopheles gambiae
ttatcagtaagt

SEQ ID NO: 122 - DNA, 12 bp, Anopheles gambiae
atgaaggtaagt

SEQ ID NO: 123 - DNA, 12 bp, Anopheles gambiae
cttcaggttagt

SEQ ID NO: 124 - DNA, 12 bp, Anopheles gambiae
attcaggtgaga

SEQ ID NO: 125 - DNA, 12 bp, Anopheles gambiae
gcttatgtgagt

SEQ ID NO: 126 - DNA, 12 bp, Anopheles gambiae
catgcagtacgt

SEQ ID NO: 127 - DNA, 12 bp, Anopheles gambiae
ataccagtgagt

SEQ ID NO: 128 - DNA, 12 bp, Anopheles gambiae
aagacggtaggt

SEQ ID NO: 129 - DNA, 12 bp, Anopheles gambiae
cttcaggtatct

SEQ ID NO: 130 - DNA, 12 bp, Anopheles gambiae
attcaggtaaga

SEQ ID NO: 131 - protein, 49 aa, Homo sapiens
VALCTFAVYVTIDENNILDAQTAFVSLALFNILRFPLNILPMVISSLVQ

SEQ ID NO: 132 - protein, 49 aa, Homo sapiens
VSVVTFSVYVLVDSNNILDAQKAFTSITLFNILRFPLSMLPMMISSMLQ

SEQ ID NO: 133 - protein, 49 aa, Homo sapiens
VTLITLWVYVYVDPNNVLDAEKAFVSVSLFNILRLPLNMLPQLISNLTQ

SEQ ID NO: 134 - protein, 51 aa, Drosophila melanogaster
ITFVTLGVYVWLHRDQEFDLNASRLFSSLALFQQLTVPLLIFPITVPIIIA

SEQ ID NO: 135 - protein, 73 aa, Homo sapiens
IAVFGYSMAVSIGGILASRCLHVDLLHSILRSPMSFFERTPSGNLVNRFSKELDTVDSMIPEVIKMFMGSLFN

SEQ ID NO: 136 - protein, 73 aa, Homo sapiens
IFVFIAHFWSAFGFVHASNILHKQLLNNILRAPMRFFDTTPTGRIVNRFAGDISTVDDTLPQSLRSWITCFLG

SEQ ID NO: 137 - protein, 73 aa, Homo sapiens
FLVMLAAMAMAAGGIQAARVLHQALLHNKIRSPQSFFDTTPSGRILNCFSKDIYVVDEVLAPVILMLLNSFFN

SEQ ID NO: 138 - protein, 69 aa, Drosophila melanogaster
VSTPAGQYAGCNARRNLHDKLLQTILHKTLHFFQVTPLGRIVNRFSNDMAVIDKKIAATGQFTLRLLQL

SEQ ID NO: 139 - protein, 72 aa, Artificial Sequence
  Description of Artificial Sequence Consensus sequence for ABC transporter membrane spanning domain
LLQGSFYLGERLGQRLRKRLFRALLRQILGLFMSFFDTNSTGELTSRLTNDVSKIRDGLGEKLGLLFQSLAT

(19) United States
(12) Patent Application Publication
(10) Pub. No.: US 2004/0244066 A1
(43) Pub. Date: Dec. 2, 2004

(54) Multidrug resistance proteins in Drosophila and Anopheles

(76) Inventors: Charles W. Roth, Rueil Malaison (FR); Paul T. Brey, Paris (FR); Inge Holm, Clamart (FR); Marine Grailles, Meung/Loire (FR); Andrey Rzhetsky, New York, NY (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/807,466

(22) Filed: Mar. 24, 2004

Related U.S. Application Data
(63) Continuation-in-part of application No. 10/667,891, filed on Sep. 23, 2003.
(60) Provisional application No. 60/413,469, filed on Sep. 26, 2002.

(51) Int. Cl. A01K 67/033; C07H 21/04; C12N 5/06; C07K 14/435

(52) U.S. Cl. 800/8; 435/69.1; 435/320.1; 435/348; 530/350; 536/23.5

(57) ABSTRACT

The invention provides, inter alia, isolated nucleic acid molecules that comprise nucleic acids encoding multiple drug resistance proteins from Drosophila melanogaster or Anopheles gambiae, herein referred to as dMRP or gMRP, respectively, vectors encoding dMRP or gMRP, and host cells transformed with vectors containing these nucleic acids.

### CROSS-REFERENCE TO RELATED APPLICATIONS

This application is a continuation-in-part of prior application Ser. No. 10/667,891, filed Sep. 23, 2003. This application is based on and claims the benefit of U.S. Provisional Application Ser. No. 60/413,469, filed Sep. 26, 2002 (Attorney Docket No. 03495.6079), and U.S. application Ser. No. 10/667,891, filed Sep. 23, 2003 (Attorney Docket No. 03495.0294), all of which are relied upon and incorporated by reference herein.

### BACKGROUND OF THE INVENTION

This invention relates to recombinant DNA technology. In particular, this invention concerns the cloning of nucleic acids encoding multiple drug resistance proteins of Drosophila melanogaster and Anopheles gambiae. More particularly, this invention provides isolated nucleic acid compounds encoding multiple drug resistance proteins of Anopheles gambiae and Drosophila melanogaster. Vectors and transformed host cells comprising the multiple drug resistance-encoding DNA of Anopheles gambiae and Drosophila melanogaster are also provided. The invention further provides assays, which utilize these transformed host cells.

The multidrug-resistance associated protein, MRP1, a large (190 kDa) membrane glycoprotein, was identified in 1992 in a human small-cell lung cancer cell line where its overexpression conferred resistance to a large spectrum of drugs (Cole et al., 1992). MRP joined the P-glycoprotein MRP1, the original protein associated with broad resistance, discovered in 1976 (Juliano & Ling, 1976). The description of MRP1 was followed by the characterization of several other structurally related human proteins: MRPs 2 to 8; followed by MRP9 (Bera et al., 2001; Buchler et al., 1996; Dean et al., 2001; Hopper et al., 2001). These proteins, like MRP1, are members of the ATP-binding cassette (ABC) superfamily, present from bacteria to man, and involved in the energy-dependent transmembrane transport of a variety of molecules, ranging from inorganic ions to large polypeptides. Like other ABC-transporters, MRP homologs are well conserved in evolution and have been described in several mammals (Buchler et al., 1996; Kool et al., 1997; van Aubel et al., 1998), in the nematode Caenorhabditis elegans (Broeks et al., 1996), in the protozoan parasite Leishmania (Essodaigui et al., 1999), in yeast (Szczypka et al., 1994), and in plants (Lu et al., 1997).

ABC transporters are typically composed of two membrane spanning domains (MSDs), containing several transmembrane a-helices, and two cytosolic nucleotide binding domains (NBDs), responsible for the hydrolysis of ATP, thus providing the necessary energy for substrate transport. MRP1 is one of several members of the MRP subfamily characterized by a third MSD of unknown function at the N-terminus of the protein (Borst et al., 2000). The NBDs are highly conserved and share two sequence motifs, designated “Walker A” and “Walker B”, with other nucleotide binding proteins. These sequences are separated by a stretch of about 120-170 amino acids, including a short (12-13 amino acid) peptide motif called the ABC transporter “signature” region. In contrast, the MSDs are highly divergent and are probably involved with the protein's substrate specificity.

Notwithstanding these advances in the art, there continues to be a need in the art to identify MRPs in other species. For example, the identification of MRPs in insects could aid in the development of more effective insecticides.

### SUMMARY OF THE INVENTION

This invention describes the identification and characterization of a Drosophila MRP encoding a protein whose deduced amino acid sequence is closely related to that of human MRP1. (See FIG. 6.) Of particular interest is the identification of multiple copies of two exons (4 and 8), suggesting alternative splicing at the mRNA level. This hypothesis is supported by a RT-PCR strategy presented here.

The dMRP of Drosophila melanogaster was the first description of a MRP homologue in an insect. Its deduced amino acid sequence is close (48% identity and 64.5% similarity) to that of the human MRP1 discovered in 1992 (Cole et al., 1992), and involved in the drug resistance of several tumors. These proteins belong to the ABCC1 subfamily of ATP-binding cassette (ABC) transporters, known to transfer a large variety of compounds across the cell membrane. Despite their close relatedness with other subgroups of ABC transporters, such as P-glycoproteins, CFTR, and sulfonylurea receptors, MRP proteins clearly form a separate cluster within this group, thus constituting an independent family (Borst et al., 1999).

Extending these earlier findings, this invention also involves the discovery of the arrangement as a cluster of genes of the four copies of MRP homologues in Anopheles gambiae, located on the chromosome 3R arm, and that all read on the same sense. This invention shows by RT-PCR that these potential genes are all transcribed in the adult mosquito, and seem ubiquitous in the different tissues. But their respective expressions are not always equivalent. The newly described genes are called gMRP1a, gMRP1b, gMRP1c, and gMRP1d. (See FIG. 6.) The three last have a close exon-intron structure (especially gMRP1c and gMRP1d), and share a strong homology and similarity, at the level of their amino acid sequences. In contrast, gMRP1a is much more different from the others regarding its sequence as well as structure, with only three introns in place of five in gMRP1b, and 6 in gMRP1c and gMRP1d.

The invention provides, inter alia, isolated nucleic acid molecules that comprise nucleic acids encoding multiple drug resistance proteins from Drosophila melanogaster or Anopheles gambiae, herein referred to as dMRP or gMRP, respectively, vectors encoding dMRP or gMRP, and host cells transformed with vectors containing these nucleic acids.

In another embodiment, the invention provides a method for determining the insecticidal MRP inhibition activity of a compound which comprises:

a) placing a culture of insect cells, transformed with a vector capable of expressing dMRP or gMRP, in the presence of:

(i) an insecticidal agent to which said insect cell is resistant, but to which said insect cell is sensitive in its untransformed state;

(ii) a compound suspected of possessing insecticidal MRP inhibition activity; and

b) determining the insecticidal MRP inhibition activity of said compound by measuring the ability of the insecticidal agent to inhibit the growth of said insect cell.

In still another embodiment, the present invention relates to strains of Drosophila melanogaster or Anopheles gambiae in which the dMRP or gMRP gene is disrupted or otherwise mutated such that the dMRP or gMRP protein is not produced in said strains.

In yet another embodiment, the present invention relates to a method for identifying new insecticidal compounds.

This invention further provides isolated polynucleotides that comprise an isolated DNA sequence encoding SEQ ID NOS: 1, 2, 3, 4, or 5, or a vector containing the isolated polynucleotide.

The isolated polynucleotide of the invention, or a portion thereof, can be labeled with a detectable moiety.

A host cell containing the vector of the invention is also provided.

A method for constructing a transformed host cell capable of expressing SEQ ID NOS: 1, 2, 3, 4, or 5 comprises transforming a host cell with a recombinant DNA vector that comprises an isolated DNA sequence of the invention.

A method for expressing SEQ ID NOS: 1, 2, 3, 4 or 5 in a transformed host cell comprises culturing the transformed host cell of the invention under conditions suitable for gene expression.

### BRIEF DESCRIPTION OF THE DRAWINGS

This invention will be described in greater detail in the drawings in which:

FIG. 1. Comparison of deduced D. melanogastercDNA SD07655 and human MRP1 amino acid sequences. The two amino acid sequences were aligned using ClustalW. Identical residues are marked with shading. The transmembrane regions are noted by a fine underline and the ATP-binding domains are noted by a bold underline. The amino acids derived from exons 4 and 8 of the dMRP gene are presented in bold characters. The small vertical lines above and below the amino acids denote the exon junctions with the type of splice junction marked by a number noting the class: 0, 1 or 2. The dMRP amino acid sequence differs from that of sequence AY069827 at the following positions: L/V pos. 124, M/L pos. 318 and lIT pos. 448.

FIG. 2. Genomic organization of dMRP and alignment of the splice junctions with dMRP cDNA SD07655. The intron-exon organization of dMRP is illustrated at the bottom with the exons indicated by the vertical bars and introns by the thin horizontal lines. The regions of the dMRP mRNA encoded by each exon are shown at the top. Superimposed on the mRNA is a schematic of the protein with membrane spanning domains (MSDs), and nucleotide binding domains (NBDs). Within each MSD, the transmembrane helices are shown as black bars. Walker A and Walker B are designated as A and B, respectively, and C indicates the ABC family signature.

FIG. 3. Expression of exons 4 and 8 variants in cellular RNA. Total cDNA was prepared as described in Materials and Methods. A first nonspecific PCR amplification of the exon 8 (A) or 4 (B) was generated using primers in neighboring exons. The presence of cDNA from a specific exon was tested using one nonspecific primer and a second primer that represented a specific sequence in the exon being tested. All of the primer sequences and the predicted product sizes are listed in Table 2. (A) The exon 8 region of the cDNA was amplified using primers DR17/DR18. The specific amplifications used primer rev8 and the specific primer 8a-8g. (B) The MRP exon 4 region of the cDNA was amplified with primers Dr2 for and Dr6rev. The exon 4 specific reactions used primer sets Dr2for/Dr4arev (lanes A, B, C) or Dr2for/Dr4brev (lanes D, E, F). The DNA targets were the product of reaction Dr2for/Dr6rev (lanes A and D), cDNA SD07655 (lanes B and E), Anopheles gambiae genomic DNA (lanes C and F). The PCR products were separated on a 1% agarose gel using the molecular weight marker (MW) SmartLadder SF (Eurogentec; Seraing, Belgium).

FIG. 4. Amino acid alignment of dMRP variable exon 4 (A) and 8 (B) encoded peptides with the cognate peptides from other organisms. The variant dMRP peptide sequence and the equivalent sequences from Drosophila sulfonylurea receptor (Dsur, NG_{—}000795) and three human MRPs (MRP1, NM_{—}004996; MRP2, NP_{—}005836; and MRP3, Y17151) were aligned using ClustalW. Pfam refers to pfamOO664, a consensus sequence for ABC transporter Membrane Spanning Domains. Gaps were introduced to maximize sequence identity and are shown by a horizontal dash. Residues that are identical in at least half of the sequences have their background shaded and those present in more than half of the sequences are listed in the consensus (Cons). (C) Dendrogram constructed with the data of part (B) of the Figure (see infra for details).

FIG. 5. Schematic representation of the four gMRP genes showing overall organization of exons and introns. Exons appear in black and are numbered under the sequence, while introns appear in white and are numbered in italic above the sequence.

FIG. 6. Comparison of deduced A. gambiae gMRP1 a-d, Drosophila melanogasterdMRP, and human MRP1 amino acid sequences. The alignment was produced using ClustalW. Identical residues in at least half of the sequences are marked with shading. The different topological regions are indicated in bold and italic above the sequences, and are delimitated by vertical bars. MSD1-3, Membrane Spanning Domains 1 to 3; Lo, cytoplasmic loop; NBD1-2, Nucleotide Binding Domain, Linker, region linking the two halves of the protein. Walker A and Walker B are indicated as A and B, and their sequences are marked in bold, as well as the signature (C) of ABC transporters. The vertical lines in bold inside the amino acid sequences denote the exon junctions. Where several genes shared the same site, this one was emphasized by a delimitating box.

### DETAILED DESCRIPTION OF THE INVENTION

At the beginning of 2001, an international consortium organized the A. gambiae genome project, which now comes in line with the sequencing of the Plasmodium and the human genomes. Before the release of the whole sequence on (a web site) preliminary work on a BAC clone library was generated by Frank H. Collins, Univ. of Notre Dame, USA. The BAC library inserts ends were sequenced by the Genoscope (Evry, France) and representing the anopheline genome, allowed the finding of four copies of homologues of the Drosophila and human MRPs, which were further characterized. The study of the Drosophila gene had put in light a new splicing process in comparison to previously examined MRPs in several animals. Two exons are present as multiple copies, which potentially allows the insect to express as many as 14 different variants of the protein. This peculiar process was not retrieved in A. gambiae.

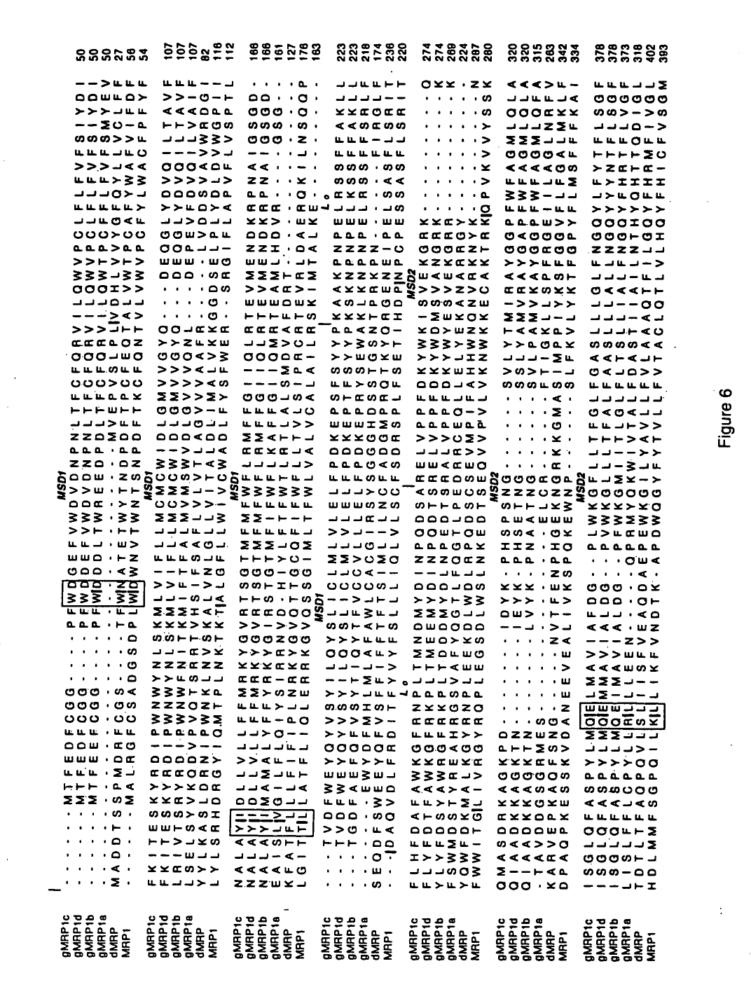

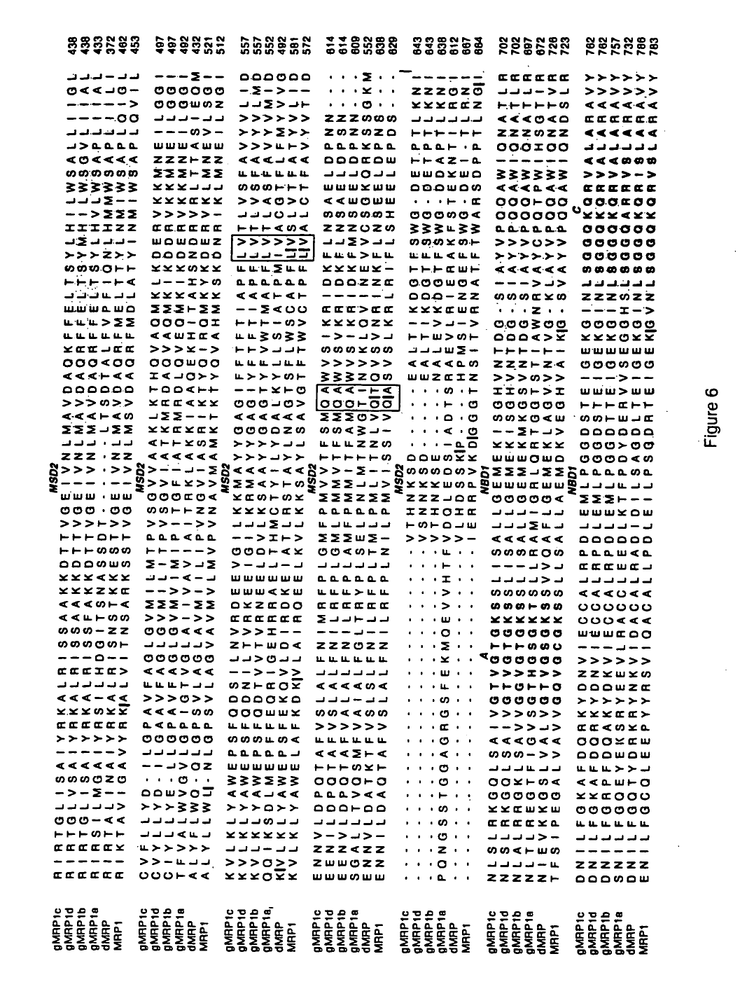

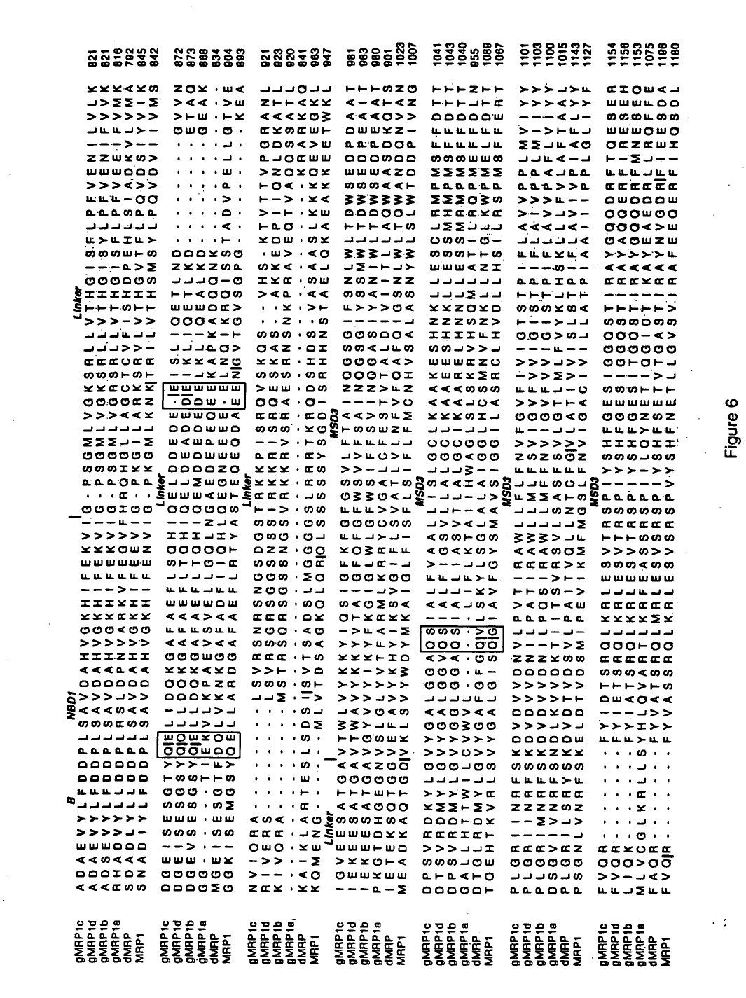

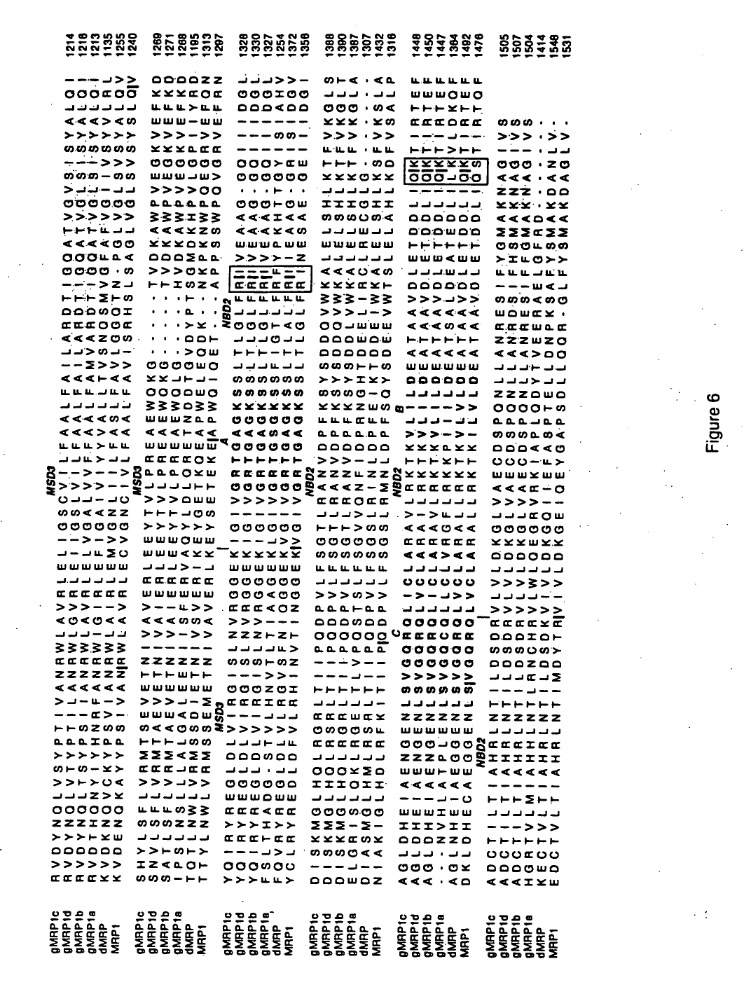

The present invention provides isolated nucleic acid molecules that comprise a nucleic acid sequence encoding dMRP or gMRP. The amino acid sequence of the protein encoded by dMRP or gMRP is provided in the Sequence Listing as SEQ ID NO: 3 and SEQ ID NOs: 2-5, respectively. As shown in FIG. 6, gMRP1a, gMRP1b, gMRP1c, and gMRP1d are identified as SEQ ID NOS: 2-5, respectively. SEQ ID NO: 1 identifies dMRP, and MRP1 is identified as SEQ ID NO: 6.

Those skilled in the art will recognize that the degenerate nature of the genetic code enables one to construct many different nucleic acid sequences that encode the amino acid sequences of SEQ ID NO: 1-5. Consequently, the constructions described below and in the accompanying examples for the preferred nucleic acid molecules, vectors, and transformants of the invention are illustrative and are not intended to limit the scope of the invention. All nucleotide and amino acid abbreviations used in this disclosure are those accepted by the United States Patent and Trademark Office as set forth in 37 C. F. R. §1.822(b) (1994).

The term “vector” refers to any autonomously replicating or integrating agent, including but not limited to plasmids, cosmids, and viruses (including phage), comprising a nucleic acid molecule to which one or more additional nucleic acid molecules can be added. Included in the definition of “vector” is the term “expression vector”. Vectors are used either to amplify and/or to express deoxyribonucleic acid (DNA), either genomic or cDNA, or RNA (ribonucleic acid), which encodes dMRP or gMRP, or to amplify DNA or RNA that hybridizes with DNA or RNA encoding dMRP or gMRP.

The term “expression vector” refers to vectors, which comprise a transcriptional promoter (hereinafter “promoter”), and other regulatory sequences positioned to drive expression of a DNA segment that encodes dMRP or gMRP. Expression vectors of the present invention are replicable DNA constructs in which a DNA sequence encoding dMRP or gMRP is operably linked to suitable control sequences capable of effecting the expression of dMRP or gMRP in a suitable host. Such control sequences include a promoter, an optional operator sequence to control transcription, a sequence encoding suitable mRNA ribosomal binding sites, and sequences that control termination of transcription and translation. DNA regions are operably linked when they are functionally related to each other. For example, a promoter is operably linked to a DNA coding sequence if it controls the transcription of the sequence, or a ribosome binding site is operably linked to a coding sequence if it is positioned so as to permit translation.

The term “MRP inhibition activity” refers to the ability of a compound to inhibit the MRP activity of a host cell, thereby increasing the insecticidal activity of an insecticidal compound against said host cell.

In the present invention, dMRP or gMRP may be synthesized by host cells transformed with vectors that provide for the expression of DNA encoding dMRP or gMRP. The DNA encoding dMRP or gMRP can be the natural sequence or a synthetic sequence or a combination of both (“semi-synthetic sequence”). The in vitro or in vivo transcription and translation of these sequences results in the production of dMRP or gMRP. Synthetic and semi-synthetic sequences encoding dMRP or gMRP can be constructed by techniques well known in the art. See Brown et al. (1979) Methods in Enzymology, Academic Press, N.Y., 68:109-151. dMRP or gMRP-encoding DNA, or portions thereof, can be generated using a conventional DNA synthesizing apparatus, such as the Applied Biosystems Model 380A, 380B, 394 or 3948 DNA synthesizers (commercially available from Applied Biosystems, Inc., 850 Lincoln Center Drive, Foster City, Calif. 94404).

Owing to the natural degeneracy of the genetic code, the skilled artisan will recognize that a sizable yet definite number of nucleic acid sequences can be constructed that encode dMRP or gMRP. All such nucleic acid sequences are provided by the present invention. These sequences can be prepared by a variety of methods and, therefore, the invention is not limited to any particular preparation means. The nucleic acid sequences of the invention can be produced by a number of procedures, including DNA synthesis, cDNA cloning, genomic cloning, polymerase chain reaction (PCR) technology, or a combination of these approaches. These and other techniques are described by Maniatis, et al., Molecular Cloning: A Laboratory Manual, Cold Spring Harbor Press, Cold Spring Harbor Laboratory, Cold Spring Harbor, N.Y. (1989), or Current Protocols in Molecular Biology (F. M. Ausubel et al., 1989 and supplements). The contents of both of these references are incorporated herein by reference.

In another aspect, this invention provides the genomic DNA encoding dMRP or gMRP, which may be obtained by synthesizing nucleic acids encoding the desired portion of SEQ ID NOS: 1-5 or by following the procedure described in the Examples.

To effect the translation of dMRP or gMRP-encoding mRNA, one inserts the natural, synthetic, or semi-synthetic dMRP or gMRP-encoding DNA sequence into any of a large number of appropriate expression vectors through the use of appropriate restriction endonucleases and DNA ligases. Synthetic and semi-synthetic dMRP or gMRP-encoding DNA sequences can be designed, and natural dMRP or gMRP-encoding nucleic acid can be modified to possess restriction endonuclease cleavage sites to facilitate isolation from and integration into these vectors. Particular restriction endonucleases employed will be dictated by the restriction endonuclease cleavage pattern of the expression vector utilized. Restriction enzyme sites are chosen so as to properly orient the dMRP or gMRP-encoding DNA with the control sequences to achieve proper in-frame transcription and translation of the dMRP or gMRP molecule. The dMRP or gMRP-encoding DNA must be positioned so as to be in proper reading frame with the promoter and ribosome binding site of the expression vector, both of which are functional in the host cell in which dMRP or gMRP is to be expressed.

Expression of dMRP or gMRP can be accomplished in cells, such as Saccharomyces cerevisiae. Suitable promoter sequences for use with yeast hosts include the promoters for 3-phosphoglycerate kinase (found on plasmid pAP12BD (ATCC 53231) and described in U.S. Pat. No. 4,935,350, Jun. 19, 1990) or other glycolytic enzymes, such as enolase (found on plasmid pAC1 (ATCC 39532)), glyceraldehyde-3-phosphate dehydrogenase (derived from plasmid pHcGAPC1 (ATCC 57090, 57091)), hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Inducible yeast promoters have the additional advantage of transcription controlled by growth conditions. Such promoters include the promoter regions for alcohol dehydrogenase 2, isocytochrome C, acid phosphotase, degradative enzymes associated with nitrogen metabolism, metallothionein (contained on plasmid vector pCL28XhoLHBPV (ATCC 39475), U.S. Pat. No. 4,840,896), glyceraldehyde 3-phosphate dehydrogenase, and enzymes responsible for maltose and galactose utilization (GALL found on plasmid pRY121 (ATCC 37658) and on plasmid pPST5, described below). Suitable vectors and promoters for use in yeast expression are further described by R. Hitzeman et al., in European Patent Publication No. 73,657A. Yeast enhancers, such as the UAS Gal enhancer from Saccharomyces cerevisiae (found in conjunction with the CYC1 promoter on plasmid YEpsec—hl 1 beta, ATCC 67024), also are advantageously used with yeast promoters.

A variety of expression vectors useful in the present invention are well known in the art. For expression in Saccharomyces, the plasmid YRp7, for example, (ATCC-40053, Stinchcomb et al., 1979, Nature 282:39; Kingsman et al., 1979, Gene 7:141; Tschemper et al., 1980, Gene 10:157) is commonly used. This plasmid contains the trp gene, which provides a selection marker for a mutant strain of yeast lacking the ability to grow in tryptophan, for example ATCC 44076 or PEP4-1 (Jones, 1977, Genetics 85:12).

Expression vectors useful in the expression of dMRP or gMRP can be constructed by a number of methods. For example, the cDNA sequence encoding dMRP or gMRP can be synthesized using DNA synthesis techniques, such as those described above. Such synthetic DNA can be synthesized to contain cohesive ends that allow facile cloning into an appropriately digested expression vector. For example, the cDNA encoding dMRP or gMRP can be synthesized to contain NotI cohesive ends. Such a synthetic DNA fragment can be ligated into a NotI-digested expression vector such as pYES-2 (Invitrogen Corp., San Diego Calif. 92121).

The techniques involved in the transformation of yeast cells, such as Saccharomyces cerevisiae cells, are well known in the art and may be found in such general references as Ausubel et al., Current Protocols in Molecular Biology (1989), John Wiley & Sons, New York, N.Y. and supplements. The precise conditions under which the transformed yeast cells are cultured is dependent upon the nature of the yeast host cell line and the vectors employed.

Nucleic acid, either RNA or DNA, which encodes dMRP or gMRP, or a portion thereof, is also useful in producing nucleic acid molecules useful in diagnostic assays for the detection of dMRP or gMRP mRNA, dMRP or gMRP cDNA, or dMRP or gMRP genomic DNA. Further, nucleic acid, either RNA or DNA, which does not encode dMRP or gMRP, but which nonetheless is capable of hybridizing with dMRP or gMRP-encoding DNA or RNA is also useful in such diagnostic assays. These nucleic acid molecules can be covalently labeled by known methods with a detectable moiety, such as a fluorescent group, a radioactive atom, or a chemiluminescent group. The labeled nucleic acid is then used in conventional hybridization assays, such as Southern or Northern hybridization assays, or polymerase chain reaction assays (PCR), to identify hybridizing DNA, cDNA, or RNA molecules. PCR assays can also be performed using unlabeled nucleic acid molecules. Such assays can be employed to identify dMRP or gMRP vectors and transformants and in in vitro diagnosis to detect dMRP or gMRP-like mRNA, cDNA, or genomic DNA from other organisms.

Compounds with demonstrated insecticidal activity can be potentiated by an MRP inhibitor such that the insecticidal activity of these compounds is extended to previously resistant species. To identify compounds useful in such combination the present invention provides an assay method for identifying compounds with Anopheles gambiae or Drosophila melanogaster MRP inhibition activity. Host cells that express dMRP or gMRP provide an excellent means for the identification of compounds useful as inhibitors of Anopheles gambiae or Drosophila melanogaster MRP activity. Generally, the assay utilizes a culture of a cell transformed with a vector that provides expression of dMRP or gMRP. The expression of dMRP or gMRP by the host cell enables the host cell to grow in the presence of an insecticidal compound to which the cell is sensitive to in the untransformed state. Thus, the transformed yeast cell culture is grown in the presence of i) an insecticidal agent to which the untransformed cell is sensitive, but to which the transformed host cell is resistant, and ii) a compound that is suspected of being an MRP inhibitor. The effect of the suspected MRP inhibitor is measured by testing for the ability of the insecticidal compound to inhibit the growth of the transformed cell. Such inhibition will occur if the suspected Anopheles gambiae or Drosophila melanogaster MRP inhibitor blocks the ability of dMRP or gMRP to prevent the insecticidal compound from acting on the cell. The Drosophila Schneider cell can be used for example. The Drosophila Schneider cell line, for example, can be used.

Oligonucleotides are provided by this invention, which are specifically hybridizable with nucleic acids encoding the dMRP and gMRP. Also provided are methods of using the oligonucleotides of the invention in methods of modulating the expression of MRP genes, inhibition of which leads to inhibition of the synthesis of dMRP and gMRP, and thereby inhibits cellular multidrug resistance. Such inhibition is desirable for preventing or modulating the development of multidrug resistance in an insect. Modified derivatives of the oligonucleotides of the invention, such as chimeras and conjugates (e.g., of an oligonucleotide and a lipophilic moiety, such as cholesterol), are also contemplated by the invention. The biological activity and cellular uptake of oligonucleotides is enhanced by such modifications.

In accordance with the present invention oligonucleotides are provided, which specifically hybridize with nucleic acids encoding an dMRP or gMRP. Certain oligonucleotides of the invention are designed to bind either directly to mRNA transcribed from, or to a selected DNA portion of, dMRP orgMRP, thereby modulating the amount of protein translated from dMRP orgMRP mRNA or the amount of mRNA transcribed from dMRP or gMRP, respectively.

Oligonucleotides can comprise nucleotide sequences sufficient in identity and number to effect specific hybridization with a particular nucleic acid. Such oligonucleotides are commonly described as “antisense.” Antisense oligonucleotides can be used as research tools and diagnostic aids. MRP genes encoding dMRP or gMRP are particularly useful for this approach. Inhibition of the expression of such MRP genes leads to inhibition of the synthesis of dMRP or gMRP and thereby inhibits cellular multidrug resistance. Such inhibition is desirable for preventing or modulating the development of multidrug resistance in an insect. The specific hybridization exhibited by the oligonucleotides of the present invention can be used for assays, purifications, cellular product preparations and in other methodologies that will be appreciated by persons of ordinary skill in the art.

Methods of modulating the expression of dMRP and gMRP comprising contacting insects with oligonucleotides specifically hybridizable with an MRP gene are herein provided. These methods are useful as a consequence of the association between MRP expression and the multidrug resistance of cells. These methods are also useful as tools, for example, in the detection and determination of the role of dMRP and gMRP expression in various cell functions and physiological processes and conditions, and for the diagnosis of conditions.

The present invention employs oligonucleotides for use in antisense inhibition of the function of RNA and DNA encoding proteins. The present invention also employs oligonucleotides that are designed to be specifically hybridizable to DNA or messenger RNA (mRNA) encoding dMRP and gMRP and ultimately modulating the amount of such proteins transcribed from their respective MRP genes. Such hybridization with mRNA interferes with the normal role of mRNA and causes a modulation of its function in cells. The functions of mRNA to be interfered with include all vital functions, such as translocation of the RNA to the site for protein translation, actual translation of protein from the RNA, splicing of the RNA to yield one or more mRNA species, and possibly even independent catalytic activity which may be engaged in by the RNA. The overall effect of such interference with mRNA function is modulation of the expression of such dMRP and gMRP. In the context of this invention, “modulation” means either an increase (stimulation) or a decrease (inhibition) in the expression of a gene. In the context of the present invention, inhibition is the preferred form of modulation of gene expression.

Oligonucleotides comprise nucleotide sequences sufficient in identity and number to effect specific hybridization with a particular nucleic acid. For example, antisense oligonucleotides, which are able to inhibit gene expression with exquisite specificity, are useful for elucidating the function of particular genes, for example, to distinguish between the functions of various members of a biological pathway. This specific inhibitory effect has, therefore, been harnessed for research use.

The oligonucleotides in accordance with this invention preferably comprise from about 8 to about 30 nucleotides. It is more preferred that such oligonucleotides comprise from about 15 to 25 nucleotides.

The present invention employs oligonucleotides targeted to nucleic acids encoding dMRP and gMRP, and oligonucleotides targeted to nucleic acids encoding such proteins. Kits for detecting the presence or absence of MRP expression can also be prepared. Such kits include an oligonucleotide targeted to an MRP gene encoding a dMRP and gMRP. Such kit and assay formats are known in the art.

“Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other.

Thus, “specifically hybridizable” and “complementary” are terms which are used to indicate a sufficient degree of complementarity or precise pairing such that stable and specific binding occurs between the oligonucleotide and the DNA or RNA target. It is understood in the art that an oligonucleotide need not be 100% complementary to its target DNA sequence to be specifically hybridizable. An oligonucleotide is specifically hybridizable when binding of the oligonucleotide to the target DNA or RNA molecule interferes with the normal function of the target DNA or RNA to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment, or in the case of in vitro assays, under conditions in which the assays are performed.

### Drosophila meloganster

Materials and methods

Genomic DNA Extraction

Adult D. melanogaster(1.5 g) were homogenized in liquid nitrogen. After addition of 7 ml of lysis buffer (100 mM Tris pH 8, 50 mM NaCl, 50 mM EDTA,1% SDS, 0.15 mM spermine, and 0.5 mM spermidine) plus proteinase K (100 μg/ml), the homogenate was mixed slowly at 37° C. for 2H. Following extraction with phenol (1×), with chloroform (1×) and dialysis overnight at 40C against TEN buffer (500 mM Tris pH 8, 10 mM EDTA, 10 mM NaCl), the DNA was treated with RNase (DNase free, 100 ng/ml) at 37° C. for 1H. It was then extracted once with phenovchloroform (1:1), with chloroform, and dialyzed overnight at 40C against TE buffer (10 mM Tris pH 8, 1 mM EDTA). The DNA was then precipitated with 1/10 volume of 3 M sodium acetate and two volumes absolute ethanol, collected, redissolved, and its final concentration was estimated on an agarose gel. DNA was prepared from larvae and pupae by the same method.

Southern Blots

DNA (10 μg) was digested with either BamHI or HindIII and the fragments were separated by electrophoresis on a 0.8% agarose gel. Following transfer to Hybond-N nylon membrane and fixation, hybridization was carried out at 650C (in 1% BSA, 0.25 M NaH₂PO₄ pH 7.2,1 mM EDTA, 150 μg/ml salmon sperm DNA) with a PCR-derived dMRP probe covering 378 bases (forward primer: GATCCGTTTATTTCCTTGCCGC; reverse primer: TCCAGGGCAGTGATTACCAGT). After hybridization, the blot was washed (in 40 mM NaH₂PO₄ pH 7.2,1% SDS, and 1 mM EDTA) 1×at RT and 2×at 65 C°.

Reverse Transcriptase-PCR (RT-PCR)

Extraction of Total RNA and cDNA Synthesis

Total RNA was isolated from 0-4H eggs, 12-24H eggs, pupae, and adult

D. melanogaster using Tri Reagent (Sigma) according to the manufacturer's instructions. The cDNA was synthesized in a 20 μl reaction mix containing 10 μg of total RNA, 1×AMV reverse transcriptase buffer (Promega), 10 U of AMV reverse transcriptase (Promega), 40 U of RNasin (Promega), 4 μg of random hexanucleotide primers (Genset), and 1 μM of each dNTP (Pharmacia). The reaction was incubated at 37° C. for 1H, followed by 95° C. for 5 min.

Amplification of Exon 4

The first PCR used primers Dr2for/Dr6rev (Table 2) and 1 μl of cDNA as template. The cycling conditions were 94° C. (3 min) for 1 cycle; 94° C. (45 sec), 60° C. (30 sec) and 72° C. (90 sec) for 35 cycles; 72° C. (10 min) for 1 cycle. This amplified product was used as template (0.5 μl) for a second PCR with the same cycling conditions with primer set specific for exon 4a or 4b (Table 2).

Amplification of Exon 8

Essentially the same conditions as those described for exon 4 were used for exon 8 containing cDNA, except that the first primer set was DR17/DR18 (Table 2) and the annealing temperature was 55° C. After electrophoresis on a 1% agarose gel, the amplified DNA was excised, purified using a Jetsorb kit (Genomed) and used as template for a second PCR with the same cycling conditions but with the exon 8 primer sets designated in Table 2.

DNA Sequencing

PCR-amplified DNA was purified using the Qiaquick PCR purification kit (Qiagen) and sequenced using an ABlprism 310 automated DNA sequencer. The cDNA clone SD07655 was sequenced directly from the plasmid after amplification in E. coli DH5a bacteria and extraction with a Qiafilter Plasmid Midi kit (Qiagen). Kits were used according to the manufacturer's instructions.

Sequence Analysis

The BLAST searches were done using the Washington University (Gish, W., 1996-2002<<http://blast.wustl.edu>>) and the NCBI (Altschul et al., 1997) versions of BLAST. ClustalW (Higgins & Sharp, 1988) or PileUp from the Wisconsin Package programs licensed from the Genetics Computer Group (GCG) was used for sequence alignments. The TMAP and Needle programs, used for transmembrane domain search and pairwise sequence comparisons, are part of the EMBOSS software package from EMBL. The software, except for the GCG package, were accessed through the Institut Pasteur web site at <http//:bioweb.pasteur.fr>. Many common DNA and protein sequence manipulations were done with DNA Strider, a Macintosh DNA program (Marck, 1988).

Characterization of the Drosophila MRP

Before the release of the annotated Drosophila genome, the amino acid sequence of human MRP1 was compared to the available genomic sequences using the TBLASTN program through the Berkeley Drosophila Genome Project (BDGP) web site (http://www.fruitfly.org). The BAC clone AC005819 was identified as containing a sequence that could code for a protein having high similarity with the human MRP1. The concerned region of the clone was then scanned against the available Drosophila Expressed Sequence Tags (ESTs), and several cDNA sequences were detected. Clone SDO7655 was selected, obtained from Research Genetics (Huntsville, Ala., USA) and sequenced. During the preparation of this report, BDGP has independently released the SD07655 cDNA sequence, accession number AY069827, for gene FBgn0032456. The gene encoding SD07655 has been given the name CG6214 by the BDGP.

When the deduced protein sequence of SD07655 was compared to those of all predicted Drosophila proteins, it naturally was most similar to the predicted product of gene CG6214, the gene from which SD07655 was transcribed. The next best predicted protein, the CG14709 product, has a blastp bit score of only 747 compared to the 2,937 score for that of CG6214. Likewise, if the human MRP1 amino acid sequence is used as the query, the bit score with our deduced SD07655 amino acid sequence (1,484) is twice as high as with the next best sequence derived from gene CG14709 (770). These results argue that CG6214 is a unique gene and is the Drosophila homolog the most related to human MRP1. This proposal was confirmed by a Southem blot of BamHI— or HindIII-digested genomic adult fly DNA probed with a SD07655 PCR product (data not shown).

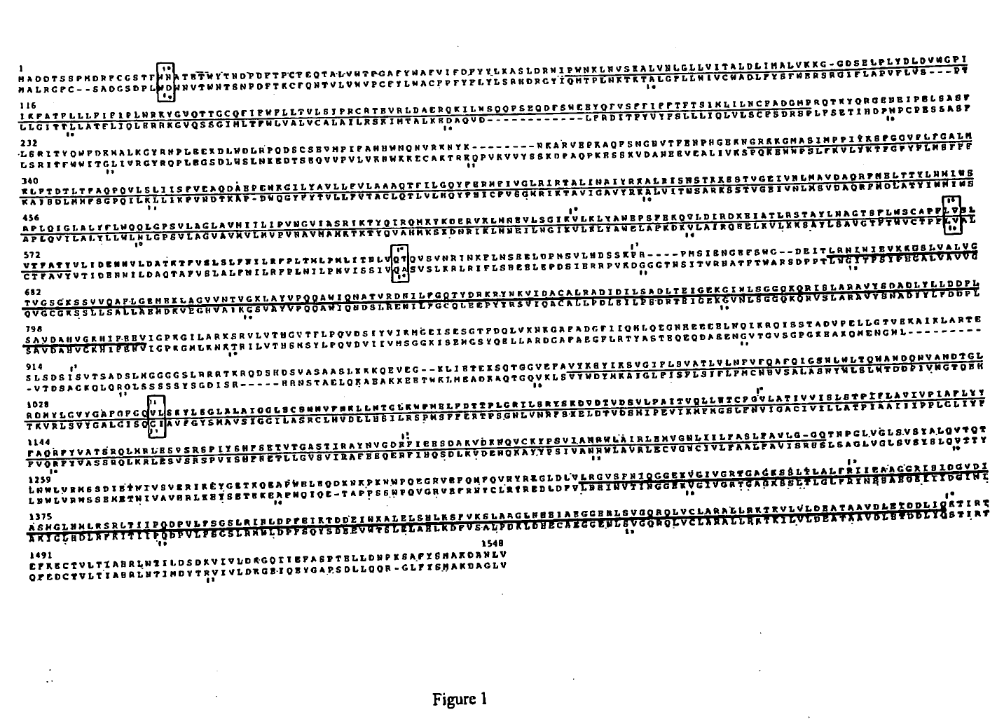

The deduced 1548 amino acid sequence generated from the SD07655 nucleotide sequence is shown in FIG. 1. The actual initiation methionine is not known, but the selected one was chosen based on a positional comparison with animal members of the MRP family, and because it was preceded by stop codons in all three ORFs, as well as having a consensus A at position −3 and a G at position +4, as discussed by Kozak (1996). The deduced amino acid sequence of this Drosophila cDNA produced an alignment with 48% identity and 64.5% similarity with the human MRP1 protein, and more than 44% identity and 60% similarity with several other MRPs (Table 1).

TABLE 1
Identity and similarity between dMRP and some other MRPs^{a}

| | Percent identity/similarity | | | | |
|---|---|---|---|---|---|
| | MRP1 | MRP2 | MRP3 | CeMRP1 | Dsur |
| dMRP | 48/64.5 | 44.7/63.4 | 47.7/63.6 | 44.1/61.2 | 23.7/38.0 |
| MRP1 | | 48.7/67.0 | 57.1/74.1 | 46.7/64.6 | 24.5/38.3 |
| MRP2 | | | 47.2/66.3 | 42.3/61.6 | 23.4/37.8 |
| MRP3 | | | | 44.3/63.1 | 24.1/38.0 |
| CeMRP1 | | | | | 24.4/38.1 |

^{a}Percent identity and similarity between the sequence pairs was calculated using the Needle program. The amino acid sequences are as follows: dMRP, D. melanogaster, MRP1, human (NM_004996); MRP2, human (NP_005836); MRP3, human (Y17151); CeMRP1, C. elegans (AB023045); Dsur, D. melanogaster sulfonylurea receptor (NG_000795).

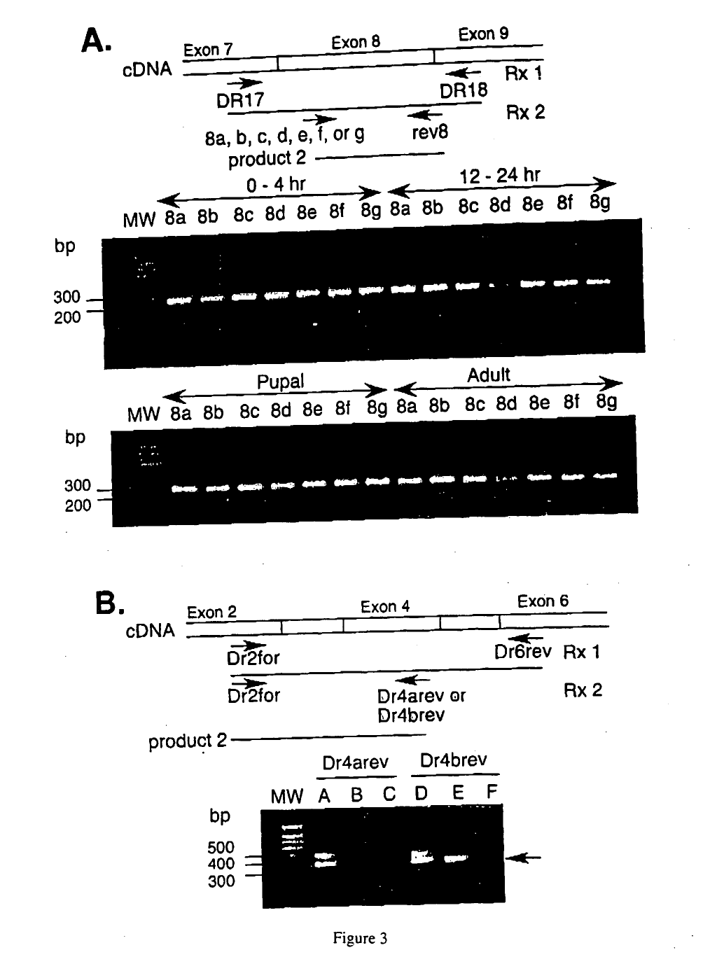

Topology predictions and comparison with other characterized MRPs point to six main regions on the mRNA, corresponding to the following parts of the protein: three MSDs (MSD 1-3), two NBDs (containing motifs A, C, B), and a linker region connecting NBD1 to MSD3 (FIGS. 1 and 3). The NH2-proximal MSD1 could span the membrane three times, while MSDs 2 and 3 may each contain four transmembrane helices, and precede NBDs 1 and 2, respectively. As seen in the alignment with the human MRP1 (FIG. 1), the similarity is especially prominent in the NBDs (706-855 and 1368-1527) and to a lesser extent in MSDs 2 and 3 (355-619 and 1020-1281). By contrast, MSD1 (110-254) and the linker region, between NBD1 and MSD3, are poorly conserved, a usual feature in MRPs. The Walker A and B boxes are easily identified in the NBDs, as well as the ABC signature. As with other MRPs, the first NBD of dMRP is 13 amino acids smaller than that of P-glycoproteins (Cole et al., 1992; Deeley & Cole, 1997).

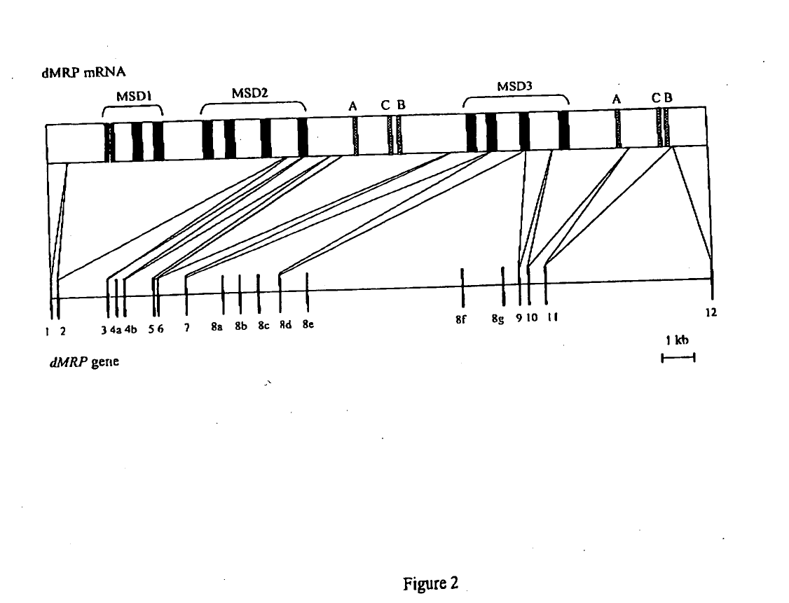

A simple comparison of the SD07655 cDNA and the genomic sequences defined a gene that spans slightly more than 22 kbp and contains 12 exons. Closer examination showed that exons 4 and 8 are represented by two and seven similar potential exons, respectively. FIG. 2 graphically depicts the predicted relationship between the cDNA and genomic sequences. This arrangement predicts a 12 exon cDNA including one copy each of the exon 4 and 8 variants as is seen with cDNA SD07655. The only important differences between this model and the FlyBase annotation are the inclusion of two copies of exon 4 in their predicted sequence, plus the predicted use of exon 8a rather than exon 8d, present in cDNA SDO7655. Their use of exon 8a is justified by the occurrence of EST LD28149, which begins in that exon sequence.

TABLE 2
PCR Primers and Their Expected Product Sizes

| | | | Size of PCR | Size of PCR | Size of PCR |
|---|---|---|---|---|---|
| | | | product | product | product |
| SEQ ID | Primer | | expected with | expected with | expected with |
| NO: | name | Primer sequence | Dr2for (bp) | DR17 (bp) | rev8 (bp) |
| 29 | Dr2for | AGTGATTGCCAGTCGCATCA | | | |
| 30 | Dr6rev | GCCGTTCTCAATGCTCATTG | 493 | | |
| 31 | Dr4arev | CTCGGCTATGTCAACACTCA | 382 | | |
| 32 | Dr4brev | TTGCACCAGGTTGGTGATCA | 382 | | |
| 33 | DR17 | AACGATCAAAATGTCGCC | | | |
| 34 | DR18 | CACGAATAGTCGATGCTCC | | 500 | |
| 35 | rev8 | GGGAATTCGCGTGGACAGACTAAT | | | |
| 36 | 8a | GGGAATTCGCGACGAACTTCTTCTC | | | 269 |
| 37 | 8b | GGGAATTCTTACCTCGTACTTCTTTTG | | | 269 |
| 38 | 8c | GGGAATTCTTGTTACAGGGTATCTATC | | | 269 |
| 39 | 8d | GGGAATTCCTATCCAAATATTTATCGGGG | | | 269 |
| 40 | 8e | GGGAATTCGTTTCACGTCATTCTTTTC | | | 272 |
| 41 | 8f | GGGAATTCGTCTTTGCAATTACGGCGC | | | 269 |
| 42 | 8g | GGGAATTCGTGTGCTAGCCTACTTTGC | | | 269 |

To assess which of the predicted exon 4 and 8 variants are used in mRNA, their expression was measured by a nested RT-PCR technique (Materials and methods, section 2.3). FIG. 3 shows that all seven alternative versions of exon 8, and the two variants of exon 4 were detected at all life stages tested, suggesting that use of the different exons is not developmentally regulated. Importantly, the sizes of the PCR products are those expected if only one copy of each exon is included in the cDNA. The accuracy of the amplification was verified by purification and sequencing of the products. In the case of exon 4, each product had a band slightly larger than the main band after the second PCR, even when the first PCR product was purified. Although we can not explain this phenomenon, sequencing the total product in both directions unequivocally resulted in the predicted sequence, either 4a or 4b.

Analysis of intron-exon boundaries

To determine the intron-exon borders, the cDNA and genomic sequences were compared. The sizes of the introns and exons and their locations are shown in Table 3 along with the intron-exon junction sequences. The size of the exons varies from 85 bp for exon 5 to 1,512 bp for exon 2, with an average around 340 bp. The mean size for the introns is 867 bp, the largest being introns 13 and 18 which are 4,965 bp and 4,791 bp, respectively. The shortest intron is number 17 with 59 bp. Compared to the human MRP1 organization, the Drosophila exons are larger and the introns smaller (Grant et al., 1997). The intron-exon boundaries (Table 3) are in accordance with those of other eukaryotic genes, especially those of human MRP1. The acceptor is characterized by a (t/c)ag sequence in 84% of the cases, exons 4a and 8b being the only exceptions with an aag sequence. The splice donor sequences are G/gt(g/a)a(g/t), with a G in 83%, gt(g/a) in 100%, a in 78%, and (g/t) in 83% of the cases. Of the 18 introns, 13 (72%) are class 0, 2 (11%) are class 1, and 3 (17%) are class 2. In comparison, MRP1 introns are class 0 in 63% of the cases, 10% are class 1, and 27% are class 2. This clearly demonstrates a mutual bias toward class 0 introns in these genes, though MRP1 uses more class 2 introns than dMRP. The importance of this difference is difficult to interpret, nevertheless, it is interesting to note that four splice junctions and their class are conserved between the two genes (FIG. 1). The variable exon 4, located just before the NBD1 region, has been conserved between the two genes based on both their splice junctions, conserved amino acid sequence and exon length. Also, the initial dMRP exon 8 splice junction has been conserved in human MRP1 despite the relative divergence in this region.

TABLE 3
Intron-exon organization of the Drosophila dMRP gene

| Exon | | | | | Intron | | |
|---|---|---|---|---|---|---|---|
| n° | Size (bp) | 3′ acceptor^{a} | exon location^{b} | 5′ donor | n° | Phase | Size (bp) |

| | | | | | | | |
|---|---|---|---|---|---|---|---|
| 1 | 181 | | −127•54 | TTCTGG/gtgagt | 1 | 0 | 74 |
| 2 | 1512 | gaacag/AACGCA | 129•1640 | ATTAAG/gtgagt | 2 | 0 | 135 |
| 3 | 138 | acatag/GTGCTC | 1776•1913 | TTCCTG/gtaaga | 3 | 0 | 128 |
| 4a | 147 | acaaag/GTTTCC | 2042•2188 | GCCGAG/gtacag | 4 | 0 | 146 |
| 4b | 147 | ttttag/GTTTCA | 2335•2481 | GTGCAA/gtaagt | 5 | 0 | 800 |
| 5 | 85 | gaatag/ACGCAA | 3282•3366 | CTAAAC/gtaaga | 6 | 1 | 62 |
| 6 | 820 | atacag/CCCATC | 3429•4248 | TTCCAT/gtaagt | 7 | 2 | 67 |
| 7 | 371 | ttttag/CTCCGT | 4316•4686 | GCCAAG/gtaagt | 8 | 1 | 904 |
| 8a | 221 | ttctag/TCGCGA | 5591•5811 | TATATG/gtaatt | 9 | 0 | 336 |
| 8b | 221 | tcgaag/TTGTTA | 6148•6368 | TTTGCG/gtaatt | 10 | 0 | 385 |
| 8c | 221 | ttccag/TTACCT | 6754•6974 | TTTGCG/gtaaat | 11 | 0 | 525 |
| 8d | 221 | atgcag/TGCTAT | 7500•7720 | TTCGGG/gtaaag | 12 | 0 | 691 |
| 8e | 224 | tcccag/GTGTGC | 8412•8635 | TTTATG/gtattt | 13 | 0 | 4965 |
| 8f | 221 | agctag/GTCTTT | 13605•13825 | TTTCAG/gtaatc | 14 | 0 | 1141 |
| 8g | 221 | tcgcag/GTTTCA | 14967•15187 | TTCGAG/gtaatt | 15 | 0 | 340 |
| 9 | 218 | ggttag/GTTCTG | 15528•15745 | AGATCG/gtatgt | 16 | 2 | 64 |
| 10 | 507 | cttcag/CTTTAT | 15810•16316 | GTTCAG/gtaagc | 17 | 2 | 59 |
| 11 | 382 | atttag/AATAAT | 16376•16757 | ATTCAG/gtgggt | 18 | 0 | 4791 |
| 12 | 393 | ctatag/AAAACC | 21549•21941 | | | | |

Comparison of the Variable Exon Encoded Sequences

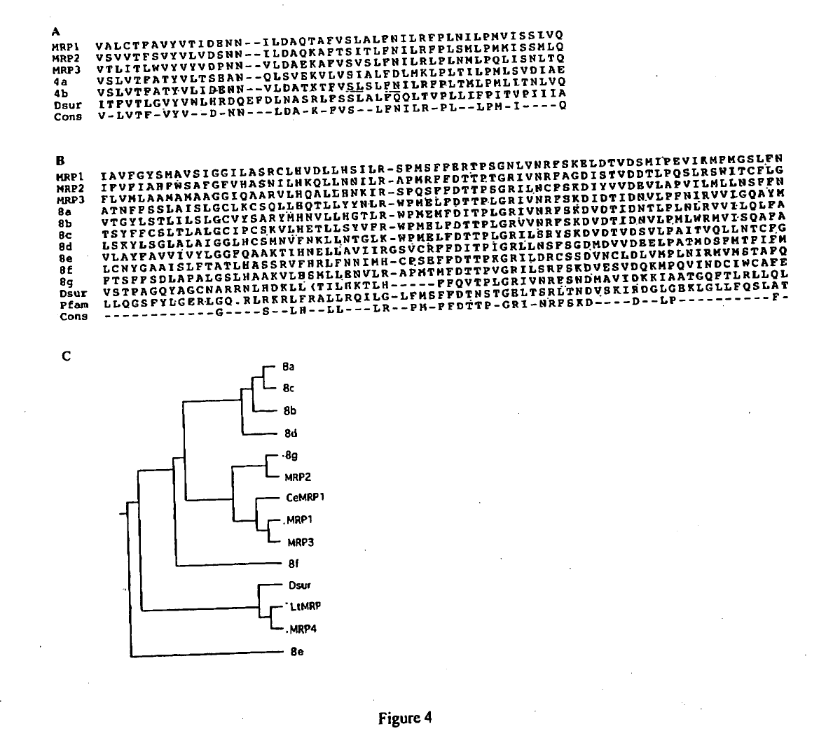

Six of seven exon 8 variants encode a 74 amino acid peptide while exon 8e encodes one additional amino acid. Likewise, the two exon 4 variants are the same length and use the same splice junction types. Alignment of the amino acid sequences encoded by the different exon 4 variants with the corresponding sequences from human MRPs 1-3 and the D. meanogaster sulphonylurea receptor (SUR) are shown in FIG. 4A. The two dMRP peptides are nearly identical in their N-terminal third, but the 4a derived peptide varies extensively from both the 4b and the human sequences in the otherwise highly conserved middle third of the peptide. This pattern suggests that conserved amino acids 18-37 may be important in normal MRP function and that the variation in this region of 4a gives the protein new properties.

When exon 8 variation is examined (FIG. 4B), there is a highly conserved region from amino acids 30-61 with the most divergence in variant 8e. The “FF” motif at positions 36-37 in the alignment is highly conserved in all the human sequences as well as in SUR and Pfam, the Protein family database consensus sequence (Bateman et al., 2002), but is only maintained in variants 8e and 8f. On the other hand, the exon 8 variants favor a Leu or Met in place of the second Phe. The extremities of the region are relatively divergent amongst all the proteins, suggesting that sequence conservation is less important in these regions.

To clarify the level of sequence similarity between the exon 8 variants and the corresponding region of other related proteins, a dendrogram was constructed using the UPGMA method on a matrix calculated with the PUZZLE program (Strimmer & von Haeseler, 1996). The dendrogram (FIG. 4C) shows the exon 8 heterogeneity: variants 8a, 8b, 8c, and 8d group together while 8g clusters with MRP2, and 8f and 8e are separated from the others. The Figure shows that while overall the Drosophila protein is very similar to human MRP1, in the exon 8 encoded region there is a great deal of diversity. This diversity could be related to the different substrates encountered by these transporters in their respective environments.

Conclusions

While alternative splicing has previously been described in the human MRPs, those cases differ from the type of splicing described in this article since previous reports detail the deletion of a terminal exon from the major product (Fromm et al., 1999; Grant et al., 1997) or the use of alternate transcriptional start sites (Bera et al., 2001; Suzuki et al., 2000). In the case of dMRP, the variants are produced by the “choice” of one exon among several possibilities for two internal regions of the protein. This process, novel in MRPs, could enable 14 different forms of the Drosophila protein.

Exon duplication is a mechanism for the evolution of protein function, and in the case of the dMRP the duplication and variation of exons 4 and 8 surely provide MRP proteins with new properties. These alterations currently provide a series of isoforms with limited, defined changes in the large protein scaffold. Understanding the substrates and physiology of the different isoforms should provide new information on the role of these variable regions in MRP function in the fly, but also in human (and other organisms) for which the corresponding regions should retain attention in future structure/function studies.

The capacity to produce a range of transporters with different specificities could be important for survival of the fly, by reducing the toxicity of an enlarged number of natural products encountered during its life. A function as important as protection from environmental toxins should not be based on a single molecule, but rather on a network of interacting enzymes with different reactivities. MRPs have a preference for glutathione conjugated substrates, and in insects glutathione S (GS)-transferases have been shown to be involved in insecticide metabolism (Tang & Tu, 1994; Ranson et al., 1997; Hemingway & Ranson, 2000). Thus, MRPs could act together with the GS-transferases to confer insecticide resistance, by coupling toxin/conjugate efflux (MRP) to toxin conjugation (GST). This hypothesis is in line with the synergistic effect of an overexpression of both a GS-transferase and MRP1, leading to high-level resistance to the cytotoxic action of several drugs in a human cell line (Morrow et al., 1998a; 1998b).

### Anopheles gambiae

Materials and Methods

Screening of the cDNA Library and Sequencing of the cDNAs

The cDNA library from Anopheles gambiae G3 fourth instar larvae in A Zap express vector (Stratagen) was kindly provided by Dr Hans-Michael Muller. 300,000 independent clones were transferred to Hybond N+membranes (Amersham) and screened for gMRP1a-d by use of two PCR derived probes specific to gMRP1a (primer set gMRP1for/gMRP1 rev, 645 bp) and gMRP1b-d (primer set MRP104/MRP105, 773 bp). Nucleotide sequences of the primers are shown in Table 4.

TABLE 4
Sequences of primers used in study

| SEQ | | | |
|---|---|---|---|
| ID | | | Sequence |
| NO: | Primer name | Primer sequence | specificity |
| 43 | gMRP1for | GGAGCGATTGTGATTTAC | gMRP1a |
| 44 | gMRP1rev | CTGGCCAACGGAAAGGTT | gMRP1a |
| 45 | MRP104 | GGTCGAGTTCAAGGACTATC | gMRP1b-d |
| 46 | MRP105 | TGAGTCACACTCCGCTACCA | gMRP1b-d |
| 47 | gMRP2for | GGAGCTCTGGTTGTGTTC | gMRP1b |
| 48 | gMRP3for | GGAAGTTGTGTCATCTTG | gMRP1c |
| 49 | gMRP4for | GGCAGCTTGGTGATCCTG | gMRP1d |
| 50 | gMRP234rev | TGACCGACCGAGAGGTT | gMRP1b-d |
| 51 | Actinf | CACACAGCATTTTTACAGCT | Actin |
| 52 | Actinr | TCTTTCATTGCCGCCGTATT | Actin |

Probes were ³²P-labeled (ICN) by random priming, and the library was screened following the Stratagen Zap express vector kit instructions. Forty eight positive plaques were purified by two rounds of screening and identified as containing gMRP1a, b, c or d by PCR with primers specific to each sequence (gMRP1for/gMRP1rev, gMRP2for/gMRP234rev, gMRP3for/gMRP234rev, and gMRP4for/gMRP234rev, respectively; Table 4). The size of the cDNA inserts was analysed by PCR with T7 and T3 universal primers. The cDNA inserts from seven positive clones that were longer than 5 kbp were sub-cloned into the pBK-CMV vector by in vivo excision from the recombinant A Zap express vector following the manufacturer's instructions. Four cDNA inserts representing each of the gMRPs were then sequenced by Genome Express (Paris, France). Sequence analysis

The ClustalW (Higgins & Sharp, 1988) program was used for sequence alignments. Tmap and Predictprotein were used for transmembrane domain search and Needle for pairwise sequence comparisons. Tmap and Needle are part of the EMBOSS software package from EMBL. The BLAST searches were done using the Washington University version of BLAST (Altschul et al., 1997). The softwares, except for the Predictprotein server (http://dodo.bioc.columbia.edu/pp), were accessed through the Institut Pasteur web site (http//:bioweb.pasteur.fr). Extraction of total RNA and cDNA synthesis

Total RNA was isolated from adult mosquitoes using Tri Reagent (Sigma) according to the manufacturer's instructions. Total RNA from tissues (salivary glands, Malpighi tubules, digestive tract from midgut to hindgut, heads and thorax) was isolated using a protocol conceived for small tissue amounts: tissues were homogenized in liquid nitrogen before dissolution in 200 μl of RNABle (Eurobio). Twenty μl of chloroform were added and samples were incubated on ice for 15 min after vortexing. They were centrifuged 45 min at 15000 g and 4° C. Eighty μl of supernatant were recovered and mixed to two volumes of 100% EtOH. After 2 min of incubation at RT, they were centrifuged 5 min at 15000 g. Pellet was washed in 180 μl of 70% EtOH, and finally recovered in 22 μl of DEPC H₂O. The cDNA was synthesized in a 20 μlreaction mix containing 10 μg of total RNA, 1×AMV reverse transcriptase buffer (Promega), 10 U of AMV reverse transcriptase (Promega), 40 U of RNasin (Promega), 4 μg of random hexanucleotide primers (Genset), and 1 μM of each dNTP (Pharmacia). The reaction was incubated at 37° C. for 1H, and stopped at 95° C. for 5 min.

Amplification on cDNA

Primers used for specific amplification of gMRP1a-don total cDNA are shown in Table 4. The cycling conditions were 94° C. (2 min) for 1 cycle; 94° C. (1 min), 56° C. (1 min 30 sec) and 72° C. (2 min) for 35 cycles (except with the actin primers, for which only 25 cycles were processed); 72° C. (10 min) for 1 cycle. Identification of A. gambiae MRPs

Before the consortium was decided on the systematic study of the A. gambiae genome, sequences already available at the Genoscope (Evry, France) were used in a comparison to the human MRP1 protein sequence, and the entirely sequenced BAC clone 22C14, containing four successive sequences with similarity to that of the human were so identified. This BAC had been previously mapped to chromosome arm 3R, on position 30D. Sequencing of the cDNAs and analysis of intron-exon boundaries

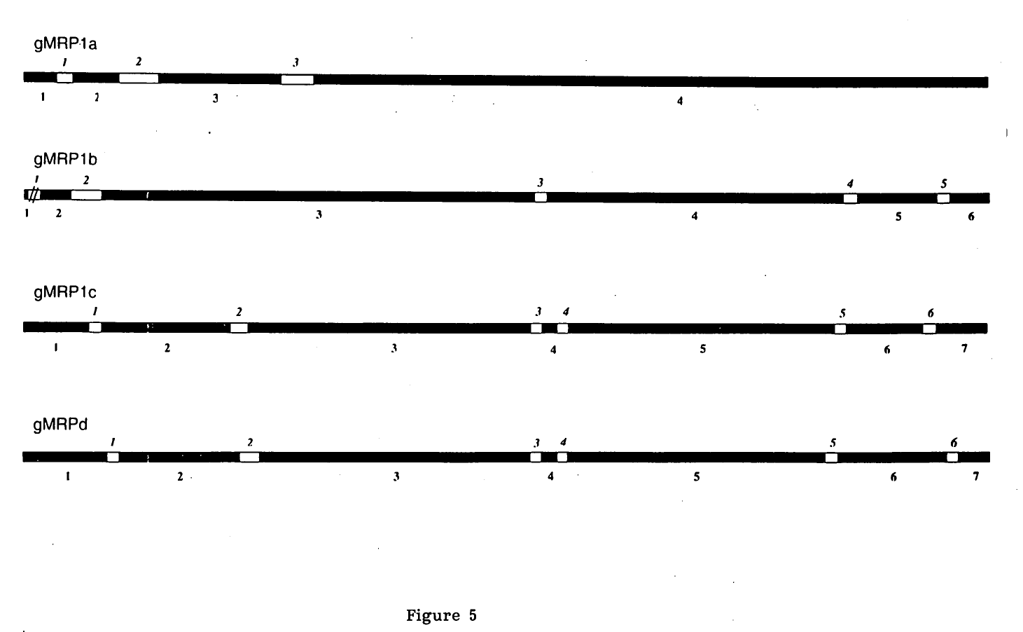

In view of identifying cDNAs specific to each of the four gMRP genes, a cDNA library of Anopheles gambiae was screened as described above. The cDNAs selected from the library were systematically sequenced for comparison with the genomic sequence available from BAC 22C14. The resulting gene structure for each gMRP is shown schematically in FIG. 5. Length of exons and introns, their location on the gene and intron-exon junctions are shown in Table 5.

The sizes proposed for the first exons are only putative, as they correspond to the longest cDNA found in the library, which appears consistent with a clustal W alignment of deduced amino acid sequences of several MRPS, and presents a favorable environment for the putative first methionine (Kozak, 1996).

TABLE 5
Organization of exon-intron junctions in the gMRPs

Exon

| | |
|---|---|
| Location on protein | Intron |

| | | | | | | | |
|---|---|---|---|---|---|---|---|
| Name | sequence^{a} | Size (bp) | 3′ acceptor^{b} | 5′ donor^{b} | Name | Phase | Size (bp) |

| | | | | | | | | |
|---|---|---|---|---|---|---|---|---|
| MRP1a | 1 | | 165 | | CCCTTG/gtgaga | 1 | 0 | 83 |
| | 2 | 10 | 234 | gtacag/GTGGAC | TCCTTG/gtaagc | 2 | 0 | 202 |
| | 3 | 88 | 566 | cttcag/GTGGGC | GCTGAG/gtaagt | 3 | 2 | 224 |
| | 4 | 277 | 3638 | atatag/ATTACT | | | | |
| MRP1b | 1 | | ND^{c} | | TTTTGG/gtaagt | 1 | 0 | 603 |
| | 2 | 13 | 300 | ttacag/GACGAT | GCTTAT/gtaagt | 2 | 0 | 76 |
| | 3 | 113 | 2144 | tttcag/ATTATG | ATACCA/gtaagt | 3 | 2 | 63 |
| | 4 | 804 | 1458 | ctctag/GGAACT | CTTCAG/gtatgt | 4 | 2 | 73 |
| | 5 | 1315 | 382 | ttccag/AATTGT | ATTCAG/gtaaga | 5 | 0 | 65 |
| | 6 | 1441 | 493 | acacag/AAAACA | | | | |
| MRP1c | 1 | | 418 | | GCTTAT/gtgagt | 1 | 0 | 69 |
| | 2 | 113 | 662 | atttag/ATCGAC | GATGCA/gtaagt | 2 | 2 | 96 |
| | 3 | 334 | 1497 | ttatag/AGAACT | TTATCA/gtaagt | 3 | 2 | 61 |
| | 4 | 833 | 77 | ttttag/GGAACT | ATGAAG/gtaagt | 4 | 1 | 60 |
| | 5 | 858 | 1369 | tttcag/AAATAT | CTTCAG/gttagt | 5 | 2 | 63 |
| | 6 | 1315 | 382 | atctag/AATTGT | ATTCAG/gtgaga | 6 | 0 | 71 |
| | 7 | 1442 | 293 | ttacag/AAAACA | | | | |
| MRP1d | 1 | | ND^{c} | | GCTTAT/gtgagt | 1 | 0 | 69 |
| | 2 | 113 | 662 | atttag/ATCGAC | CATGCA/gtacgt | 2 | 2 | 110 |
| | 3 | 334 | 1497 | tcgcag/AGAAAT | ATACCA/gtgagt | 3 | 2 | 65 |
| | 4 | 833 | 80 | tttcag/ACAACT | AAGACG/gtaggt | 4 | 1 | 98 |
| | 5 | 859 | 1372 | caccag/AAATTA | CTTCAG/gtatct | 5 | 2 | 73 |
| | 6 | 1317 | 382 | ttccag/AATTGT | ATTCAG/gtaaga | 6 | 0 | 65 |
| | 7 | 1444 | 363 | ccacag/AAAACA | | | | |

^{a}The numbering is based on amino acid one being the putative first Met.
^{b}Capital letters are used for the sequence in the exon and small case letters for sequence in the intron.
^{c}Not Determined.

For gMRP1b and gMRP1d, sequences could be amplified by RT-PCR upstream of the longest cDNA, and no size was proposed for the first exon in this case The longest exon is exon 4 for gMRP1a (3638 bp), exon 2 for gMRP1b (2144 bp), exon 3 for gMRP1c (1497 bp), and exon 3 as well for gMRP1d (1497 bp). The smallest seems to be exon 1 (165 bp) for gMRP1a, exon 4 for gMRP1b (382 bp), exon 4 for gMRP1c (77 bp), and exon 4 for gMRP1d (80 bp). The mean size of exons is 1151 bp, 1119 bp, 671 pb, and 726 bp for gMRP1a, gMRP1b, gMRP1c, and gMRP1d, respectively. Introns are small in all four gMRPs, with mean sizes of 170 bp, 69 bp, 70 bp, and 80 bp for gMRP1a, gMRP1b, gMRP1c, and gMRP1d, respectively. These data contrast with those of the human MRP1 (Grant et al., 1997), for which exons are small (not greater than 311 bp), while introns are very large (from one to several kbp). Even when compared to the Drosophila structure, the gMRPs contrast by the fact that their exons are longer than their introns. Nevertheless, intron-exon boundaries share the characteristics of other eukaryotes. The acceptor sequence is characterized by a (t/c)ag motif, and the splice donor sequence is gt(g/a)ag(t/a), with a gt in 100%, (g/a) in 95%, and ag(t/a) in 74% of the cases. Introns 1 and 2 of gMRP1a are of class 0, while intron 3 is of class 2. Among the four introns in gMRP1b, two are of class 0, and 2 of class 2. Three of the six introns of gMRP1c are of class 2, two are of class 0, and one of class 1. In gMRP1d, the pattern is identical to that of gMRP1c. Interestingly, the class 2 introns are predominant in these genes, while MRP1 and dMRP have more class 0 introns. A common feature, however, is the few number of class 1 introns (only one in gMRP1c and gMRP1d, and none in gMRP1a and gMRP1b). Analysis on aligned sequences shows conservation of splice site locations and class of introns between the four gMRPs (FIG. 5), some data consistent with a common origin of these copies by duplication of an ancestor gene. Only the first splice site in gMRP1a has no equivalent in the three other gMRPs. gMRP1c and gMRP1d share exactly the same splice site, thus reinforcing the similarity between these two forms, which are also the closest at the level of their nucleotide sequence (Table 6).

(a) Capital letters are used for the sequence in the exon and small case letters for sequence in the intron.

(b) The numbering is based on nucleotide one being the A of the initiation codon.gMRP1b lacks two sites in comparison to them, and gMRP1a lacks three sites. But each of them also has one site at the 5′ end of the sequence which is not present in gMRP1c and gMRP1d. Among all these sites, some are shared by either MRP1 or dMRP, but interestingly there is only one site (the first site in gMRP1b, which is not shared by other gMRPs), which is also common to dMRP and MRP1, while these two genes also have some common splice sites. It is noteworthy that conservation inside the anopheline species and between this one and the others involves in nearly all cases splice sites that are present in gMRP1c and gMRP1d. This indication is clearly in favor of these two forms being the most related to the common ancestor of gMRPs, with gMRP1a and gMRP1b having lost some splice sites after their divergence. Identity/similarity comparisons between MRP1 and gMRP deduced amino acid sequences also show that gMRP1a and gMRP1b are the most divergent (Table 6). gMRP1a is the gene that has the most different sequence and has lost the most splice sites in comparison to the others, an observation that suggests that it has diverged the first from the primitive sequence. The second gene to be duplicated must have been gMRP1b, but based on this data, it is not clear if it is gMRP1c or gMRP1d that gave birth to the others. These two genes have exactly the same splice sites, and their identity/similarity values with other MRPs are not significantly different, so that it cannot be determined which of these two sequences is closest to those of other species. Even if gMRP1a was the first duplicated copy in the anopheline, it is difficult to explain its high degree of divergence, that leads its deduced amino acid sequence to share no more identity with other gMRPs than with those of other species. One explanation could be that this duplicated copy was able to by-pass the selection pressure controlling the ancestral gene evolution by acquiring slightly new functions, thus allowing evolution on a new way.

TABLE 6
Identity and similarity between gMRPs and some other MRPs

| | Percent identity/similarity | | | | | | | |
|---|---|---|---|---|---|---|---|---|
| | gMRP1b | gMRP1c | gMRP1d | dMRP | MRP1 | MRP2 | MRP3 | CeMRP1 |
| gMRP1a | 30.1/48.4 | 29.8/48.8 | 30.3/49.2 | 29.7/47.0 | 29.3/46.1 | 29.2/47.6 | 30.1/46.0 | 29.3/45.6 |
| gMRP1b | | 78.3/88.9 | 79.7/89.4 | 52.9/71.2 | 48.4/66.8 | 45.2/65.2 | 48.2/65.4 | 45.2/63.1 |
| gMRP1c | | | 88.2/94.4 | 53.7/70.9 | 48.7/66.8 | 45.0/64.7 | 47.1/65.4 | 44.3/62.2 |
| gMRP1d | | | | 53.0/71.2 | 48.7/66.9 | 44.5/64.6 | 45.9/65.0 | 44.5/63.1 |
| dMRP | | | | | 59.9/68.2 | 46.8/65.8 | 50.6/67.2 | 46.4/63.8 |
| MRP1 | | | | | | 48.7/65.9 | 56.9/74.0 | 46.5/63.0 |
| MRP2 | | | | | | | 46.7/65.3 | 42.2/60.4 |
| MRP3 | | | | | | | | 45.5/62.6 |

Percent identity and similarity between pairs of sequences was calculated using the Needle program, with gap opening and extension values of 5 and 1, respectively. The amino acid sequences are as follows: gMRP1a, A. gambiae; gMRP1b, A. gambiae; gMRP1c, A. gambiae; gMRP1d, A. gambiae; dMRP, D. melanogaster, MRP1, human; MRP2, human: MRP3, human: CeMRP1, C. elegans.

Characterization of the Deduced Amino Acid Sequences

The predicted amino acid sequences of the four A. gambiae MRPs deduced from the sequencing studies are shown in FIG. 6, aligned with the Drosophila dMRP and human MRP1. For any of the A. gambiae are we sure of having entire cDNAs, so we do not know with certainty which is the initiation codon; however, based on comparison of the deduced amino acid sequences, we predict that the first methionine, for each gMRP, could be the one indicated in FIG. 6. In each case, the chosen methionine is preceded by STOP codons in all three ORFs, without other identical residue (data not shown). gMRP1c and gMRP1d also share an A nucleotide in position −3, in accordance with Kozak's observations on translation start sites (1996).

Alignment of these anopheline MRPs with those of other animal organisms clearly showed a difference between data obtained for gMRP1 a and those of the three other gMRPs. gMRP1 b, c, and d share more homogeneous sequences, while gMRP1 a is strikingly different. Its identity and similarity values with other MRPs are low, even with the three other gMRPs (Table 6). The mean identity and similarity values between gMRP1a and the three other gMRPs are 30.1% and 48.8%, respectively, and they are of 29.5% and 46.5% with other MRPs. In contrast, gMRP1 b-d are much more related to one another (82.1% mean identity, and 90.9% mean similarity) than to other MRPs (48.4% mean identity and 66.2% mean similarity). Among these, dMRP is the closest (53.2% mean identity and 71.1% similarity), while among the human MRPs, it is MRP1 (51.4% mean identity and 67.2% mean similarity). Identity between MRP1 and gMRP1 b-d is even greater than between MRP1 and MRP4 or MRP5 in the human species (Borst et al., 2000). MRP3, the sequence of which is close to that of MRP1 (Borst et al., 1999) also shares great identity/similarity with the gMRP1 b-d (47.9%/65.7% respective mean values). gMRP1c and gMRP1d are those which match the best with other sequences analysed here. It is then probable that one of these two sequences gave birth to the three others by gene duplication. Despite the great divergence of gMRP1 a, the sequences of the gMRPs are more conserved between themselves than are those of the human MRPs integrated in this study (56.1% mean identity, and 69.8% mean similarity for the first ones versus 50.8% mean identity and 68.4% mean similarity for the second). The best matches between gMRPs and dMRP agrees with the logical hypothesis of a common ancestor gene for the two insects.

FIG. 6 highlights that the more conserved regions are the NBDs, the MSDs sharing less similarity. The less conserved regions are the first MSD and the linker region, as usual in ABC transporters (Hipfner et al., 1999). Walker A and B motifs, as well as the ABC signature are clearly identified on FIG. 6. gMRPs share the distinctive features of MRP1-like proteins, that are a structure with three MSDs in place of two for most of the ABC transporters, and the lack of 13 amino acids in the first NBD, in comparison to P-glycoprotein (Hipfner et al., 1999), which confirms their relatedness to MRP1 rather than to other structurally close proteins.

The deduced amino acid sequence of gMRP1c was compared to the sequences contained into the Sptrnrdb database using the BLASTP program, and the result showed best scores with SD07655 and CG6214 (1527 and 1507, respectively), which are sequences from the D. melanogaster MRP1 homologue (Grailles et al., submitted paper). The next best scores are those of the murine MRP (1338) and human MRP1 (1335). On the other hand, when the sequence was compared to the anopheline databases from TREMBL and SWISSPROT (http://konops.imbb.forth.gr/AnoDB), the highest scores observed were much lower than those cited above; they were not greater than 108 for SWISSPROT, and 67 for TREMBL. These results, in accordance with in situ hybridization, suggest that no other paralogues than those described here exist in the anopheline genome.

Comparison between the four cDNAs and the genomic sequence represented by BAC 22C14 allowed us to evaluate the length of each gene to slightly more than 5 kbp. gMRP1a-d respectively contain 2, 5, 7, and 7 exons. Contrary to the Drosophila gene dMRP, these ones do not possess exons represented as multiple interchangeable copies, and the introns are small (the biggest is only 603 bp), and these reasons explain the great difference observed between these genes length and that of the Drosophila or human homologues (22 kbp and 200 kbp, respectively). Comparison of the four copy structure and expression

In human, the predicted structures of MRP proteins distinguishes two sub-groups: the first one contains MRP1, 2, 3, 6 and 7, which possess three MSDs, while the second group, containing MRP4 and MRP5, has a classical ABC-transporter structure with 2 MSDs (Hopper et al., 2001). The topological predictions we have realized for our gMRPs are in favor of three MSDs for these proteins, which means that they may belong to the first group. This is in accordance with their sequence similarity with the genes of this group, which is more important than with those of the second group (data not shown). The combination of secondary structure predictions and biochemical analyses have led to a topological model of MRP1 with 17 transmembrane domains, five in MSD1, and six in MSDs 2 and 3 (Kast & Gros, 1998). Also, the two NBDs have been clearly localized to the intracellular compartment by several methods (Flens et al., 1994; Bakos et al., 1996; Hipfner et al., 1996, 1997; Kast & Gros, 1998). Although we have no biochemical data on the gMRPs that could help in predicting their membrane topology, we assume, in regard to their relatedness with MRP1, that they should have a similar organization. This means that the NH₂ extremity of these proteins is more probably external, and the two NBDs are internal, as well as the COOH extremity. Respecting these features imposes some constraints on the organization of the transmembrane helices. It means that MSD1 must have an odd number of transmembrane domains, while MSD 2 and 3 each have an even number of these. We tried to integrate these data on the analysis of the secondary structure predictions obtained for our sequences, using the Tmap and PredictProtein softwares. Taking into account the results of these programs and known features on MRP1 structure, the first MSD of our gMRPs could contain five transmembrane helices, while MSD2 and MSD3 could each span the membrane six times. Such predictions are in accordance with the structure predicted for.MRP1.

TABLE 7
Expression in Different Tissues

| | | Tissue | | | | |
|---|---|---|---|---|---|---|
| | | Salivary | Malpigian | Digestive | | |
| Gene | Date | gland | Tubes | Tube | Head | Thorax |
| gMRP1a | 18/10/01 | | | | + | +++ |
| | 22/10/01 | | | | 0 | +++ |
| | 25/10/01 | 0 | +++ | +++ | + | +++ |
| | 1/03/02 | +++ | ++ | +++ | 0 | |
| | 8/03/02 | ++ | +++ | +++ | ++ | ++ |
| | 22/03/02 | +++ | ++ | +++ | +++ | +++ |
| gMRP1b | 18/10/01 | | | | +++ | +++ |
| | 22/10/01 | | | | +++ | +++ |
| | 25/10/01 | 0 | ++ | +++ | ++ | +++ |
| | 1/03/02 | +++ | 0 | +++ | + | |
| | 8/03/02 | +++ | + | +++ | +++ | ++ |
| | 22/03/02 | +++ | 0 | +++ | +++ | +++ |
| gMRP1c | 18/10/01 | | | | ++ | +++ |
| | 22/10/01 | | | | + | +++ |
| | 25/10/01 | 0 | +++ | +++ | ND | +++ |
| | 21/03/01 | +++ | + | ++ | + | ++ |
| | 21/03/01 | +++ | +++ | +++ | + | |
| | 22/03/02 | +++ | + | +++ | ++ | +++ |
| gMRP1d | 18/10/01 | | | | + | +++ |
| | 22/10/01 | | | | + | +++ |
| | 25/10/01 | 0 | ++ | +++ | ND | +++ |
| | 1/03/02 | +++ | + | +++ | 0 | |
| | 8/03/02 | + | +++ | +++ | + | ++ |
| | 22/03/02 | +++ | 0 | +++ | ++ | +++ |

In an attempt to see if each of these four genes could be related to a specific function, we compared their relative expressions between different tissues. This was performed by semi-quantitative RT-PCR, as described above. The tissues used were thorax, heads, Malpighi tubules, digestive tract, and salivary glands. After extraction of the RNAs from these tissues, reverse transcription (RT) was performed, followed by PCR with primers specific to genes gMRP1a-d. Table 7 shows that cDNAs corresponding to all four genes were present in these tissues. At least four experiments were conducted on each tissue. Comparison of the relative expression of the four gMRP genes in each tissue did not show differences of expression inside the salivary glands, the digestive tracts, and the thorax. However, in the Malpighi tubules we could note a stronger expression of gMRP1a, and a lesser expression of gMRP1b. At the head level, gMRP1b was always strongly expressed, while gMRP1c and gMRP1d exhibited a week expression. Our results are in favor of an ubiquitous localization of the four gMRPs, but the differences of expression observed could be related to functional specificities acquired by gene duplication evolution.

In summary, Drosophila melanogaster has a gene very similar to human MRP1 that encodes a full ABC-transporter containing three membrane-spanning domains (MSDs) and two nucleotide-binding domains (NBDs). This 19 exon insect gene, dMRP (FBgn0032456), spans slightly more than 22 kb. The cDNA SD07655 representing this gene was sequenced and found to contain sequences from 12 exons including single copies of two exons having multiple genomic copies. The gene contains two variant copies of exon 4 and seven of exon 8. While a cDNA contains only one version of each variable exon, all forms of these variable exons were detected in adult fly mRNA. These results predict that Drosophila could make 14 different MRP isoforms from a single gene by substituting different variable exons. This is the first report of any organism using differential splicing of alternative, internal exons, to produce such a large array of MRP isoforms having the same size, but with limited and defined internal variations. Defining the functional differences in the dMRP isoforms should provide clues to the structure/function relationships of the amino acids in these MRP domains, both for the insect enzyme and for those of other species.

The invention also constitutes the description of MRP homologues found in the Anopheles gambiae mosquito by computational analysis of genomic sequences. cDNAs corresponding to these genes were produced, and compared to the genomic sequences, in view of determining their structure. The particular interest of this study is the existence, in the anopheline genome, of a cluster of four genes of the same family, two of them having very close sequences. It does not seem that other copies of this gene family exist elsewhere in the genome, and the four described here, which have probably emerged by gene duplication, could be characterized by specific physiological roles, due to some differences observed in their tissue expression.

It is now clear, in regard of the increasing knowledge on human MRPs, and also on other animals, that this family of proteins is involved in a variety of biological processes, explaining the diversity of forms encountered in the organisms. In the only other insect studied for MRPs, Drosophila melanogaster, it seems that there is a unique gene, with internal variations at the exon level. In the case of the anopheline, no such variable exons have been detected, but genetic diversity was introduced by duplication. Particularly striking is the divergence observed in the gMRP1a paralogue in comparison to the three other genes. Even if it is not clear how to interpret this fact, it can be assumed than acquisition of a new function important for the mosquito could have explained a high rate of sequence divergence from the other paralogues during evolution.

RT-PCR experiments have demonstrated the transcription of each of the four forms in the adult mosquito, in the different tissues examined. Differences were noted in the level of expression of some forms on certain tissues, and also on a mosquito cell line, were it was clearly observed that gMRP1 a and gMRP1 b were far less expressed than the two others. These observations agree with the hypothesis that the different forms should have different physiological roles.

Logically, one can expect that the physiological function of these proteins will be similar to that of the human MRP1, because their structures are close. Even closer is the D. melanogaster dMRP, and data on that protein function will probably greatly help in the study of the anopheline forms, when they are available. It has been recently shown that dMRP could transport the well known substrate MRP1, leukotriene C4 (Roger Deeley, personal communication), which is the first functional demonstration of the relationship between the drosophila and human proteins. In D. melanogaster, the existence of variable exons has led us to suspect that these regions could play an important role in the structure and/or function of the protein (results under submission). The comparison of the sequences in this region, between the fly and human MRPs, showed that some amino acids were highly variable in some otherwise conserved regions. When we compare the alignment between gMRPs, MRP1 and dMRP in the same regions, this variability is confirmed. It seems to us that functional studies on these gene products should particularly focus on these amino acids, because they could lead to different substrate affinities. As the four forms of gMRPs were not equally expressed in all tissues one can speculate that their different specificities could involve them in different biological functions. Biochemical studies should help answer these questions in the future. The region corresponding to exon 4 of dMRP has not yet been studied functionally, but in the region corresponding to exon 8 one amino acid of the human MRP1 has been shown to be important in transport of anthracyclines, vincristine, and VP16 (Zhang et al., 2001). This residue is highly polymorphic in the gMRPs, as well as another residue, corresponding to Thr¹²⁴² of MRP1, which is also important for the transport of several substrates in human. The variability at this level is probably related to functional specificity of the 4 gMRPs. Also, it is interesting to note that a near by amino acid, Trp¹²⁴⁶ of MRP1, which functional importance has been shown (Ito et al., 2001), and which is very conserved in the ABCC group (the MRPs group inside ABC transporters), is not conserved in any of the four gMRPs. Instead, gMRP1a has a Ser residue at this position, and the three others a Phe. This observation must be of physiological importance.

MRPs are transporters of organic anions, either conjugated or not to acidic ligands such as reduced glutathione (GSH), glucuronate or sulfate. Very little is currently known about the physiological function of MRPs, but the different members of the family in human have been characterized for different substrate specificities, which are most probably related to different physiological functions (Borst et al., 2000). Likewise, two homologue genes can have different substrate specificities from one species to another, which is illustrated for example by the human MRP1 and murine mrp (Stride et al., 1999). It is thus very probable that the variations observed in otherwise very conserved regions in the anopheline MRPs are related to such specificity. It is striking that, although three of the gMRPs have very similar sequences, and gene organization (especially gMRP1c and gMRP1d), the fourth, gMRP1a, is very different, in structure as well as in organization. It is too early to speculate about the implications of this observation, but is worth noting that this form exhibited different levels of expression according to the tissue or cultured cell line analysed. Probably, the different forms of anopheline MRPs act as members of a network of detoxifying proteins which clear the cell from the variety of products encountered in the environment.

One particular aspect of these protein function that has kept our attention is their strong affinity for glutathione conjugates, because it has been shown that glutathione S (GS)-transferases were involved in insecticide metabolism in the anopheline mosquito (Tang & Tu, 1994; Ranson et al., 1997; Hemingway & Ranson, 2000). It is so possible that the two proteins act together in a biological process that would couple conjugation of the toxic products to exportation of the newly formed complex. This hypothesis is supported by results on human cell lines that have shown a synergistic effect between GS-transferase and MRP1 overexpression to confer high-level resistance to several drugs (Morrow et al., 1998a; 1998b).

Gene duplication is one of the mechanisms contributing to evolution by conferring new properties to a protein, or even new function. This is more probably the case with the four gMRPs described here. Changes that have appeared in the gene sequence after the different duplications seemingly have provided functional specificity to each of the isoforms, which is relevant with the differences observed in expression at the tissue level. It now remains to be determined which are the substrates of each isoform, and how these contribute to the physiology of the mosquito; particularly interesting will be to investigate their potential role in insecticide resistance, knowing that insecticide control of vector is currently the most successful strategy against malaria (Collins et al., 2000). A better understanding of the mechanisms involved in insecticide resistance will help developing new control measures in the future.

### REFERENCES

The following references are cited herein, and the disclosure of each reference is relied upon and incorporated by reference herein.

Altschul, S. F., Madden, T. L., Schaffer, A. A., Zhang, J., Zhang, Z., Miller, W., Lipman, D. J. 1997. Gapped BLAST and PSI-BLAST: a new generation of protein database search programs. Nucl. Acids Res. 25, 3389-3402.

Bateman, A., Birney, E., Cerruti, L., Durbin, R., Etwiller, L., Eddy, S. R., Griffiths-Jones, S., Howe, K. L., Marshall, M., Sonnhammer, E. L. 2002. The Pfam protein families database. Nucl. Acids Res. 30, 276-280.

Bera, T. K., Lee, S., Salvatore, G., Lee, B., Pastan, 1.2001. MRP8, a new member of ABC transporter superfamily, identified by EST database mining and gene prediction program, is highly expressed in breast cancer. Mol. Med. 7, 509-516.

Borst, P., Evers, R., Kool, M., Wijnholds, J. 2000. A family of drug transporters: the multidrug resistance-associated proteins. J. Natl. Cancer Inst. 92, 1295-1302.

Broeks, A., Gerrard, B., Allikmets, R., Dean, M., Plasterk, R. H. A 1996. Homologues of the human multidrug resistance genes MRP and MRP contribute to heavy metal resistance in the soil nematode Caenorhabditis elegans. Embo J. 15, 6132-6143.

Büchler, M., König, J., Brom, M., Kartenbeck, J., Spring, H., Horie, T., Keppler, D. 1996. cDNA cloning of the hepatocyte canalicular isoform of the multidrug resistance protein, cMrp, reveals a novel conjugate export pump deficient in hyperbilirubinemic mutant rats. J. Biol. Chem. 271, 15091-15098.

Cole, S. P. C., Bhardwaj, G., Gerlach, J. H., Mackie, J. E., Grant, C. E., Almquist, K. C., Stewart, A. J., Kurz, E. U., Duncan, A. M. V., Deeley R. G. 1992. Overexpression of a transporter gene in a multidrug-resistant human lung cancer cell line. Science 258, 1650-1654.

Dean, M., Rzhetsky, A., Allikmets, R. 2001. The human ATP-binding cassette (ABC) transporter superfamily. Genome Res. 11, 1156-1166.

Deeley, R. G., Cole, S. P. C. 1997. Function, evolution and structure of multidrug resistance protein (MRP). Semin. Cancer Biol. 8, 193-204.

Essodaïgui, M., Frézard, F., Moreira, E. S. A., Dagger, F., Gamier-Suillerot, A. 1999. Energy-dependent efflux from Leishmania promastigotes of substrates of the mammalian multidrug resistance pumps. Mol. Biochem. Parasitol. 100, 73-84.

Fromm, M. F., Leake, B., Roden, D. M., Wilkinson, G. R., Kim, R. B. 1999. Human MRP3 transporter: identification of the 5′-flanking region, genomic organization and alternative splice variants. Biochim. Biophys. Acta 1415, 369-374.

Grant, C. E., Kurz, E. U., Cole, S. P. C., Deeley, R. G. 1997. Analysis of the intron-exon organization of the human multidrug-resistance protein gene (MRP) and alternative splicing of its mRNA. Genomics 45, 368-378.

Hemingway, J., Ranson, H. 2000. Insecticide resistance in insect vectors of human disease. Annu. Rev. Entomol. 45, 371-391.

Higgins, D. G., Sharp, P. M. 1988. CLUSTAL: a package for performing multiple sequence alignment on a microcomputer. Gene 73, 237-244.

Hopper, E., Belinsky, M. G., Zeng, H., Tosolini, A., Testa, J. R., Kruh, G. D. 2001. Analysis of the structure and expression pattern of MRP7 (ABCC10), a new member of the MRP subfamily. Cancer Lett. 162, 181-191.

Juliano, R. L., Ling, V.1976. A surface glycoprotein modulating drug permeability in Chinese hamster ovary cell mutants. Biochim. Biophys. Acta 455, 152-162.

Kool, M., de Haas, M., Scheffer, G. L., Scheper, R. J., van Eijk, M. J. T., Juijn, J. A.,

Baas, F., Borst P. 1997. Analysis of expression of cMOAT(MRP2), MRP3, MRP4, and MRP5, homologues of the multidrug resistance-associated protein gene (MRP1), in human cancer cell lines. Cancer Res. 57, 3537-3547.

Kozak M. 1996. Interpreting cDNA sequences: some insights from studies on translation. Mamm. Genome 7, 563-574.

Lu, Y. P., Li, Z. S., Rea, P. A. 1997. AtMRP1 gene of Arabidopsis encodes a glutathione S-conjugate pump: isolation and functional definition of a plant ATP-binding cassette transporter gene. Proc. Natl. Acad. Sci. USA 94, 8243-8248.

Marck, C. 1988. ‘DNA Strider’: a ‘C’ program for the fast analysis of DNA and protein sequences on the Apple Macintosh family of computers. Nucl. Acids Res. 16,1829-1836.

Morrow, C. S., Smitherman, P. K., Diah, S. K., Schneider, E., Townsend, A. J. 1998a. Coordinated action of glutathione S-transferases (GSTs) and multidrug resistance protein 1 (MRP1) in antineoplastic drug detoxification. J. Biol. Chem. 273, 20114-20120.

Morrow, C. S., Smitherman, P. K., Townsend, A. J. 1998b. Combined expression of multidrug resistance protein (MRP) and glutathione S-transferase P1-1 (GSTP1-1) in MCF7 cells and high level resistance to the cytotoxicities of ethacrynic acid but not oxazaphosphorines or cisplatin. Biochem. Pharmacol. 56, 1013-1022.

Ranson, H., Cornel, A. J., Foumier, D., Vaughan, A., Collins, F. H., Hemingway, J. 1997. Cloning and localization of a glutathione S-transferase class I gene from Anopheles gambiae. J. Biol. Chem. 272, 5464-5468.

Strimmer, K., von Haeseler, A. 1996. Quartet puzzling: A quartet maximum likelihood method for reconstructing tree topologies. Mol. Biol. Evol. 13, 964-969.

Suzuki, T., Sasaki, H., Kuh, H. J., Agui, M., Tatsumi, Y., Tanabe, S., Terada, M., Saijo,

N., Nishio, K. 2000. Detailed structural analysis on both human MRP5 and mouse mrp5 transcripts. Gene 242, 167-173.

Szczypka, M. S., Wemmie, J. A., Moye-Rowley, W. S., Thiele, D. J. 1994. A yeast metal resistance protein similar to human cystic fibrosis transmembrane conductance regulator (CFTR) and multidrug resistance-associated protein. J. Biol. Chem. 269, 22853-22857.

Tang, A. H., Tu, C. P. 1994. Biochemical characterization of Drosophila glutathione S-transferases Dl and D21. J. Biol. Chem. 269, 27876-27884. van Aubel, R. A., van Kuijck, M. A., Koenderink, J. B., Deen, P. M., van Os, C. H.,

Russel, F. G. M. 1998. Adenosine triphosphate-dependent transport of anionic conjugates by the rabbit multidrug resistance-associated protein Mrp2 expressed in insect cells. Mol. Pharmacol. 53, 1062-1067.

Bakos E., Hegedüs T., Holló Z., Welker E., Tusnády G. E., Zaman G. J. R., Flens M. J., Váradi A., and Balazs S. (1996). Membrane topology and glycosylation of the human multidrug resistance-associated protein. J. Biol. Chem. 271: 12322-12326.

Borst P., Evers R., Kool M., and Wijnholds J. (1999). The multidrug resistance protein family. Biochim. Biophys. Acta 1461: 347-357.

Collins F. H., Kamau L., Ranson H. A., and Vulule J. M. (2000). Molecular entomology and prospects for malaria control. Bull. World Health Organ. 78: 1412-1423.

Flens M. J., lzquierdo M. A., Scheffer G. L., Fritz J. M., Meijer C. J . L . M., Scheper R. J., and

Zaman G. J. R. (1994). Immunochemical detection of the multidrug resistance-associated protein MRP in human multidrug-resistant tumor cells by monoclonal antibodies. Cancer Res. 54: 4557-4563.

Higgins, D. G., and Sharp, P. M. (1988). CLUSTAL: a package for performing multiple sequence alignment on a microcomputer. Gene 73: 237-244.

Hipfner D. R., Almquist K. C., Stride B. D., Deeley R. G., and Cole S. P. C. (1996). Location of a protease-hypersensitive region in the multidrug resistance protein (MRP) by mapping of the epitope of MRP-specific monoclonal antibody QCRL-1. Cancer Res. 56: 3307-3314.

Hipfner D. R., Almquist K. C., Leslie E. M., Gerlach J. H., Grant C. E., Deeley R., and Cole S. P. C. (1997). Membrane topology of the multidrug resistance protein (MRP). J. Biol. Chem. 272: 23623-23630.

Hipfner D. R., Deeley R. G., and Cole S. P. C. (1999). Structural, mechanistic and clinical aspects of MRP1. Biochim. Biophys. Acta 1461: 359-376.

Ito K. I., Olsen S. L., Qiu W., Deeley R. G., and Cole S. P. C. (2001). Mutation of a single conserved tryptophan in multidrug resistance protein 1 (MRP1/ABCC1) results in loss of drug resistance and selective loss of organic anion transport. J. Biol. Chem. 276: 15616-15624.

Kast C., and Gros P. (1998). Epitope insertion favors a six transmembrane domain model for the carboxy-terminal portion of the multidrug resistance-associated protein. Biochem. 37: 2305-2313.

Stride B. D., Cole S. P. C., and Deeley R. G. (1999). Localization of a substrate specificity domain in the multidrug resistance protein. J. Biol. Chem. 274: 22877-22883.

Zhang D. W., Cole S. P. C., and Deeley R. G. (2001). Identification of a nonconserved amino acid residue in multidrug resistance protein 1 important for determining substrate specificity. J. Biol. Chem. 276: 34966-34974.

What is claimed is:

1. A purified polynucleotide comprising a purified nucleotide sequence encoding a sequence selected from the group consisting of the SEQ ID NOS.: 1, 2, 3, 4, or 5, or a fragment thereof.

2. A purified polynucleotide of claim 1 or a fragment thereof, which is labeled with a detectable moiety.

3. An expression vector comprising a purified polynucleotide, or one of its fragments, of claim 1 or 2.

4. A vector comprising a purified nucleotide sequence of claim 2.

5. A host cell containing the vector of claim 4.

6. A method for constructing a transformed host cell capable of expressing SEQ ID NOS: 1, 2, 3, 4, or 5 said method comprising transforming a host cell with a recombinant DNA vector that comprises an isolated DNA sequence of claim 1.

7. A method for expressing SEQ ID NOS: 1, 2, 3, 4, or 5 in a transformed host cell, said method comprising culturing said transformed host cell of claim 5 under conditions suitable for gene expression.

8. An isolated nucleic acid molecule that comprises nucleic acids encoding multiple drug resistance proteins from Drosophila melanogaster or Anopheles gambiae.

9. A method for determining the insecticidal MRP inhibition activity of a compound, which comprises:
a) placing a culture of insect cells, transformed with a vector capable of expressing dMRP or gMRP, in the presence of:
(i) an insecticidal agent to which said insect cell is resistant, but to which said insect cell is sensitive in its untransformed state;
(ii) a compound suspected of possessing insecticidal MRP inhibition activity; and
b) determining the insecticidal MRP inhibition activity of said compound by measuring the ability of the insecticidal agent to inhibit the growth of said insect cell.

10. A strain of Drosophila melanogaster or Anopheles gambiae in which the dMRP or gMRP gene is disrupted or otherwise mutated such that the dMRP or gMRP protein is not produced in said strain.

11. A composition having an insecticidal activity and comprising at least a compound, which is obtainable by expression of nucleotidic sequence corresponding to one of claims 1 or 2.
